(19) United States
(12) Patent Application Publication
(10) Pub. No.: US 2026/0266805 A1
(43) Pub. Date: Sep. 10, 2026

(54) METHOD AND COMPOSITIONS FOR ANALYZING TOXICITY IN ORGANISMS

(71) Applicant: vivoVerse, LLC, Austin, TX (US)

(72) Inventors: Adela BEN-YAKAR, Austin, TX (US); Sudip MONDAL, Pflugerville, TX (US); Adam LAING, Austin, TX (US); Amber SHEN, Round Rock, TX (US); Evan HEGARTY, Austin, TX (US); Andrew John Theodore DUPLISSIS, Austin, TX (US)

(21) Appl. No.: 19/168,624

(22) PCT Filed: Mar. 25, 2024
(86) PCT No.: PCT/US2024/021346

§ 371 Date: Sep. 24, 2025

Related U.S. Application Data
(60) Provisional application No. 63/454,444, filed on Mar. 24, 2023.

(51) Int. Cl. G01N 33/50; G01N 15/1433; G01N 33/58

(52) U.S. Cl. CPC G01N 33/5085; G01N 15/1433; G01N 33/582

(57) ABSTRACT

A method of analyzing internal components of an organism, the method comprising a) taking an image of internal components of the organism, while said internal components are contained in vivo, and b) analyzing the internal components. Also a method of determining toxicity or efficacy of a substance, the method comprising: a. exposing an organism to one or more substances of interest; b. taking one or more images of one or more internal components of the organism, c. analyzing internal components of the organism from the images to determine the effects of the substance on the organism, and; d. using the results of the analysis to determine the toxicity or efficacy of the one or more substances.

### CROSS-REFERENCE TO RELATED APPLICATIONS

This application claims the benefit of priority to U.S. Provisional Application No. 63/454,444, filed Mar. 24, 2023, which is incorporated by reference herein in its entirety.

### GOVERNMENT SUPPORT CLAUSE

This invention was made with government support under Grant Nos. 1R43ES033579-01, 5R44MH118841-01, 5R44MH118841-02, and T R43MH118841-01 awarded by the National Institutes of Health. The Government has certain rights in the invention.

### BACKGROUND

Currently, toxicology studies use large animal models such as rats, mice, rabbits, etc. For DART studies, the animals are grown and maintained for several months until they become reproductively active. The animals are then orally exposed to chemicals and monitored for their developmental phenotypes. Both males and females are administered for DART outcomes. The parents are observed during mating, pregnancy, and the termination of the study. Several offspring parameters are studied, such as the number of pups, sex of pups, stillbirths, live births, runts (pups that are significantly smaller than corresponding control pups), and the presence of gross abnormalities. In addition to developmental parameters, certain chemical studies require endpoints related to neuronal pathology, animal behavior, cognitive analysis, etc. Besides the significant ethical concern, a few more disadvantages of animal experimentation are the requirement of skilled or trained human resources, time-consuming protocols, and inbred animal species. A very high cost is involved in breeding, housing, and lengthy protocols of animal experiments. Finally, poor concordance of the DART data using a large animal model influences the toxicology field to avoid animal use and adopt new approach methodologies (NAMs), such as in silico approaches and model organisms. Several NAMs are being developed to implement model organisms (such as Drosophila, zebrafish, C. elegans, planaria, and daphnia) into toxicology research programs. The small animal models have several advantages in terms of their simple assay designs and low cost compared to the studies with the large animal models. Among these small animal models, C. elegans has been established as the most powerful genetic model. The popularity of this model, recognized by 3 Nobel prizes, has led to several discoveries as an outcome of the existing & growing toolsets such as the availability of ~2000 mutants, CRISPR-Cas9 reagents, RNAi library, and ~1000 wild isolates with diverse genetic backgrounds.

Several C. elegans toxicology studies have used optical measurements from a flow-cytometer (COPAS Biosort, Union Biometrica) to measure the body lengths of a batch of worms. The assay involves a few small numbers of hermaphrodites growing with a chemical and fed in bacterial food, starting larval 4 (L4) stages, for a few days. The animals lay ~300 embryos over 3-4 days during this time. The embryos hatch at different time points (depending on when they are laid from the adult body) in the media and are fed with bacteria. At the end of the studies, the population (including the adults and all the new progenies) is analyzed using the COPAS system for developmental phenotypes such as body size. Using the scattered light, one can measure the time-of-flight to quantify object lengths. The assay is limited to low-resolution characterization of the body size only from a mixed population of progenies hatched at different time points during the 3-4 days interval. As an alternative technology, plate-based images are captured to assess worm length to identify toxicology effects on worm development.

Another conventional method to study the progenies is to use 10-30 adults and transfer them every day to a fresh chemical solution while collecting all the progenies for observations. The next day all the eggs and hatched larvae are counted to estimate the number of laid embryos and hatching rates for a given number of adults. The work is very laborious and prone to contamination from multiple days of experiments and several manual interventions.

The existing state-of-the-art toxicology studies provide the gross developmental parameters (average body size) from all the progenies produced by a small number of parents (typically ~4 adults) and hatched over 3-4 days. The assay is limited to either low-resolution characterization of the body size or low-throughput analyses of adults using high-content manual imaging of a small number of worms.

Conventional C. elegans-based methodologies use several manual steps, demanding an open access technology, which is difficult to practice for robust and uniform chemical exposure to the animals for a reproducible concentration-dependent response. For example, volatile chemicals, volatile solvents, and UVCBs might require new methodologies with complete isolations when testing a panel of chemicals to avoid sample contamination while providing uniform exposure during the entire period.

What is needed in the art is a more effective way to carry out imaging in vivo of organisms.

### SUMMARY

Other systems, methods, features and/or advantages will be or may become apparent to one with skill in the art upon examination of the following drawings and detailed description. It is intended that all such additional systems, methods, features and/or advantages be included within this description and be protected by the accompanying claims.

Disclosed herein is a method of analyzing internal components of an organism, the method comprising a) taking an image of internal components of the organism, while said internal components are contained in vivo, and b) analyzing the internal components.

Also a method of determining toxicity or efficacy of a substance, the method comprising: a. exposing an organism to one or more substances of interest; b. taking one or more images of one or more internal components of the organism, c. analyzing internal components of the organism from the images to determine the effects of the substance on the organism, and; d. using the results of the analysis to determine the toxicity or efficacy of the one or more substances.

### BRIEF DESCRIPTION OF DRAWINGS

FIGS. 1A-1E depict embryo phenotypes within the C. elegans germline and effects of FUdR on DART phenotypes. FIG. 1A shows a diagram of the D1 C. elegans germline FIG. 1B shows 10× timelapse fluorescence microscope images of D1 dat-1::GFP (BY200) worms showing the GFP+ dopaminergic neurons of developed embryos around the vulva region of the germline at 0 seconds and +5 seconds. Green and magenta circles indicate the relative positions of sets of neurons between frames as they move within the eggshell. FIGS. 1C-1E show body length (FIG. 1C), total embryo number (FIG. 1D), and moving embryo number (FIG. 1E) of N2 (obtained from CGC) worms treated with 0-10000 μM FUdR. Hill functions were fitted and used to find the EC₅₀ for each phenotype.

FIG. 2 depicts a schematic of the assay workflow for quantifying DART phenotypes in C. elegans after exposure to test substances. Synchronized L1 larval stage C. elegans are cultured in liquid media in varying doses of a test substance or control chemical. After 72 hrs of treatment when control worms have developed to the D1 adult stage, the animals are loaded into a microfluidic-based trapping and immobilization system (the vivoChip-24x) for rapid 4D high-resolution imaging. Software-assisted image analysis is used to quantify embryonic phenotypes (human scoring of ~30-40 embryos inside ~1,000 worms taking 1-2 days) and developmental phenotypes (automated ML-based determination of body dimensions completed in >30 minutes) associated with DART toxicity.

FIGS. 3A-3F depict vivoChip-24x design and operation for high-resolution imaging obtained for DART studies. FIG. 3A shows a vivoChip-24x in its holder. FIG. 3B shows that each vivoChip-24x contains 24 wells with 40 trapping channels each (each well and its set of connected trapping channels is referred to throughout as a “device”). FIG. 3C shows N2 worms with ~40 embryos immobilized in 1 of 24 devices in a vivoChip-24x. The worms trap at different channel positions relative to the mouth of the channels due to body dimension differences. FIG. 3D shows the duration of each step of the assay and the volume of data produced. FIG. 3E shows 10× imaging of a trapped worm. FIG. 3F shows examples and schematics of embryos with different phenotypes (moving, folded, and early) and grouped phenotypes (non-moving and developed).

FIGS. 4A-4D depict acceptance criteria and quality checks for the DART assay. FIG. 4A shows that quality checks were performed at all stages of the assay from culture maintenance to imaging and analysis. FIGS. 4B-4D show box plots of adults per plate (FIG. 4B), embryos per plate (FIG. 4C), and % embryos hatched (FIG. 4D) across multiple batches of worms prepared for DART experiments. Red lines indicate the minimum acceptance criteria (AC) for each parameter.

FIG. 5 is a screenshot of vivoImager software showing automated imaging of a vivoChip-24x.

FIGS. 6A-6D depict the vivoChip-24x imaging workflow. FIG. 6A shows a flowchart of the automated imaging workflow. FIG. 6B shows that fiduciary cross markers beside every device are used to find the best focal plane. FIG. 6C shows that bounding boxes for all worms trapped in the channels are identified by a worm-finding ML model. FIG. 6D shows that five 10× obj. FOVs capture all 40 channels in 4D within the device at the ideal xy locations and best focal plane.

FIGS. 7A-7E depict an automated image acquisition and analysis pipeline to rapidly screen C. elegans models for DART studies. FIG. 7A is a flowchart showing the process of imaging data cropping from accessing the raw images and saving images of individual microfluidic channel s and metadata on the server. FIG. 7B shows example images of a worm within a single channel show the steps of automated body analysis using an ML-assisted segmentation (finding the best focus plane from the cropped stack, using the model to predict body pixels, and generating a binary mask with pixels >0.5 probability of being part of the body). FIG. 7C shows images from an individual worm showing the marking and scoring of embryos by human users. FIG. 7D shows an overview of the ML body segmentation process which predicts a body mask from the best focal plane and generates a binary image of the predicted worm body outline.

FIG. 7E shows an overview of the manual analysis process where human scorers label and categorize all embryos within the germline of each worm. Both scores are stored for each experiment in a database for plotting and statistical analysis.

FIGS. 8A-8F depict workflow for automated worm body dimension measurement using a body predicting ML model. FIGS. 8A-8B show toxicated (4 μM methylmercury) or control worms are different sizes and may be found at different locations within the tapering trapping channels. FIGS. 8C-8D show 10× cropped images of each worm (best focal plane) are analyzed by a trained U-net model to predict pixels belonging to the worm body. A 2D binary mask of the predicted worm body is generated from those pixels with >0.5 probability. Body length is measured by finding the longest skeleton after pruning. Volume is found by multiplying each pixel within the mask by the channel height at that location. FIGS. 8E-8F show body length (FIG. 8E) and body volume (FIG. 8F) of 4 μM CH₃Hg treated (blue) and H₂O control (green) populations.

FIG. 9 is a screenshot of vivoAnalyzer software.

FIGS. 10A-10B depict a correlation between scorers for total and developed embryos within the same training dataset. 4 different scorers used vivoAnalyzer to score a training dataset of worms (randomly chosen from control and toxicated worms) for developed (FIG. 10A) and total embryos (FIG. 10B) and their scores were compared among all possible scorer combinations. The average correlation coefficients (r) were 0.9199 (developed embryos) and 0.9231 (total embryos)

FIGS. 11A-11I depict variance between individual worms and technical replicates in a single experiment. FIG. 11A shows a map of the 24-well plate culturing and vivoChip imaging, showing the conditions tested. In total, 6 technical replicates were plated for each condition (one row per plate), and 6 experimental replicates were plated on different days. FIGS. 11B-11E show well-to-well variability for body length. The box and whiskers show the spread of individual worms within each well for a single representative experiment. Phenotypes shown are body length, body volume, total embryos, and developed embryos. FIGS. 11F-11I show scatter plots depicting the coefficient of variation (CV) between technical replicates (6 wells) within each 6 experiments with the black line representing the median CV of all 6 experiments and the dotted red line referring to the 30% CV as reference. CV could not be calculated for developed embryos in rows A and C as means were close to 0 as would be expected from the positive control compounds.

FIGS. 12A-12F depict well-to-well variation. The box and whiskers (left) show the spread of individual worms within each well for a single representative experiment. Scatter plots (right) show the coefficient of variation (CV) between technical replicates (6 wells) within each 6 experiments with the black line representing the median CV of all 6 experiments and the dotted red line referring to the 30% CV as reference. Phenotypes shown are early embryos (FIGS. 12A-12B), non-moving embryos (FIGS. 12C-12D), and body area (FIGS. 12E-12F).

FIGS. 13A-13D depict variance between well averages and experimental replicates performed at different days. Box and whiskers showing the spread of individual worms within corresponding wells (A01, B01, C01 & D01) in all 6 experimental replicates for body length (FIG. 13A), body volume (FIG. 13B), total embryos (FIG. 13C), and developed embryos (FIG. 13D). A01=100 μM FUdR, B01=1 μM CH₃Hg, C01=4 μM CH₃Hg, D01=H₂O.

FIGS. 14A-14C depict experiment-to-experiment variation. Box and whiskers showing the spread of individual worms within corresponding wells (A01, B01, C01 & D01) in all 6 experimental replicates for early embryos (FIG. 14A), non-moving embryos (FIG. 14B), and body area (FIG. 14C). A01=100 μM FUdR, B01=1 μM CH₃Hg, C01=4 μM CH₃Hg, D01=H₂O.

FIGS. 15A-15I depict the effect of number of worms/well, number of wells/day, and number of replicates on DART parameters. FIGS. 15A-15D show the effect of the number of channels analyzed of the coefficient of variation (CV) of body length (FIG. 15A), body volume (FIG. 15B), total embryos (FIG. 15C), and developed embryos (FIG. 15D) for both water control and 4 μM CH₃Hg treated wells. 3 experimental repeats were used for in each sample. FIGS. 15E-15H show mean phenotype values with 95% confidence intervals obtained from sampling 2-6 experimental replicate wells for body length, body volume, total embryos, and developed embryos. FIG. 15I shows average CV values from randomly selecting 3 or 6 experimental replicate wells from the dataset for the 1 μM CH₃Hg treatment or the water control. The red lines indicate the 30% acceptability threshold.

FIGS. 16A-16C depict experimental design for dose response assays and raw imaging data for propiconazole. FIG. 16A shows that each 24-well plate and vivoChip can hold 10 populations of worms treated with 10 concentrations of 2 chemicals, plus 2 wells of a positive control for DART (4 μm CH₃Hg) and 2 vehicle control wells. Chemical A in these studies was propiconazole. FIGS. 16B-16C show representative worm images from high and low concentrations of propiconazole. Colored dots mark the locations and phenotypes of moving, folded, and early embryos as marked by the scorer. The images are cropped and aligned to show relative size and trapping position relative to the channel exit.

FIGS. 17A-17I depict dose response curves for the effects of 2 agricultural chemicals using C. elegans model. The effects of propiconazole on body dimensions (body length (FIG. 17A), area (FIG. 17B), & volume (FIG. 17C)), embryonic development (early (FIG. 17D), folded (FIG. 17E), & moving embryos (FIG. 17F)), and grouped embryonic stages (developed (moving+folded) (FIG. 17G), non-moving (early+folded) (FIG. 17H), & total embryos (FIG. 17I)). EC₁₀ estimates are indicated with 95% confidence interval bands.

FIGS. 18A-18C depict automated analysis of C. elegans body using a U-Net architecture. FIG. 18A shows a schematic of the 4-layer U-Net. FIG. 18B shows an overview of the basic convolutional layer that defines the computation in the encoder and decoder. FIG. 18C shows an overview of the vision transformer (ViT).

FIG. 19 depicts that embryos inside the adult C. elegans are predicted using a 2.5D U-Net model. On top, an image of an adult worm immobilized inside the microfluidic channel is presented. The middle image is the manually scored embryos (ground truth). The bottom image is the predicted scores from the model.

FIGS. 20A-20E depict high-resolution images of the fluorescence reporter to visualize C. elegans reproductive organ. FIG. 20A shows dual-color fluorescence images of AUM1039 animals using 20×, 0.75 NA objective. The animals express a membrane-bound GFP (pie-1::gfp::PH) and a nuclear mCherry (pie-1::mCherry::his-58. FIG. 20B shows a picture of germlines from two worms and two different z-focal planes immobilized in the vivoChip. FIGS. 20C-20D show images of the germline and gonad arms (GFP signal only) showing dead and viable embryos in the C. elegans population treated with FUdR and vehicle control, respectively. FIG. 20E shows a time-lapse image of a viable embryo showing movement inside the eggshell.

FIG. 21 depicts a high-throughput assay using C. elegans to identify multiple phenotypes associated with health benefits or adverse effects from active ingredients.

FIGS. 22A-22C depict image-based studies of C. elegans studies to capture multiple phenotypes. FIGS. 22A-22B show images of C. elegans growing on standard multi-well plates. The images are shown for 0 hr, 48 hr, and 72 hr for solvent control (FIG. 22A) and positive control (FIG. 22B). FIG. 22C shows a schematic of a microfluidic device (vivoChip-24x) to immobilize ~1,000 C. elegans from 24 different populations. The right image shows the bottom of an individual well with 40 parallel immobilization channels. The bottom shows an image of all 40 channels with an immobilized worm, presented below in the picture.

FIGS. 23A-23D depict quantifying stored lipids inside the C. elegans body. FIG. 23A is a schematic showing C. elegans intestine in bright field and lipids stained with Nile red (NR). The NR signal is visible in red fluorescence images. The total NR signal increases when the amount of lipids in the worm's body increases. FIG. 23B shows images of NR-stained worms inside the microfluidic chip. The bright field image is used to identify the worm inside the channel. The worm body (predicted body mask) is detected inside the channel using an automated machine-learning algorithm. The red signal within the body is used to quantify the NR signal, reflecting the amount of stored lipid in well-fed and 4-hour-starved animals. FIG. 23C shows average NR signal from device-immobilized worms under different amounts of NR dye and incubation conditions. FIG. 23D shows the average NR signal for control worms and treatment conditions shows changes in the NR signal. The data is presented as mean±SEM. The statistical significance is calculated using a pairwise t-test to identify p-values >0.05 (ns) and p-values <0.001 (***).

FIGS. 24A-24D depict analyzing stress response in C. elegans using the vivoChip platform. FIG. 24A is a schematic showing healthy and stressed worms with low and high expression of the GFP reporter. FIG. 24B and FIG. 24D each show an image of a single worm immobilized inside the vivoChip from a vehicle control population (FIG. 24B) and H₂O₂ treated populations (FIG. 24D). Both bright-field and GFP images are shown. FIG. 24C shows average intensity for control and H₂O₂-treated worms. Data presented as mean±SEM. The statistical significance is calculated using a pairwise t-test to identify p-values <0.001 (***).

FIGS. 25A-25C depict identifying C. elegans organs and organ structures using high-resolution, bright field, and fluorescence images using the vivoChip platform. FIG. 25A shows a schematic of a C. elegans immobilized inside the vivoChip platform. The schematic represents different organs that could be identified and analyzed using image processing or automated machine learning algorithms. FIG. 25B shows average pharynx length from D1 adult animals treated with 1.6 μM chlorpyrifos (CPS) and DMSO (0.25%) starting at the L1 stage. FIG. 25C shows images of healthy and unhealthy worms treated with FITC-Dextran dye to visualize the intestinal lumen in live worms.

FIGS. 26A-26D show N2 C. elegans were treated with 0, 0.1, 0.5, 1, 5, 10, 50, 100, 500, 1000, 5000 and 10000 μM of FUdR and number of (a) total embryos, (b) moving embryos, (c) body length, and (d) midpoint body diameter found from the timelapse images. 500 μM FUdR or higher caused growth arrest at stages earlier than could be immobilized in the chip, so animals were imaged in the well plates. No accurate measurement of diameter could be taken in well plate images. Black line shows the weighted mean for all 5 experiments.

FIGS. 27A-27E show (a) Calculated power of the assay to detect an effect of a given size with an N number of 40. 80% is considered the minimum acceptable power for assays in general. (b) Comparison of all embryonic phenotypes scored across 6 experiment with the number of worms scored. (c) Individual measurements for 6 H2O control wells in a single representative experiment. 95% confidence intervals (CIs) are shown for each well in red. (d) Separation of measured values between H₂O and CH₃Hg treated populations for the moving and total embryo phenotypes, with 95% CIs. (e) Coefficients of variance for all wells and conditions in a single representative experiment.

FIGS. 28A-28H show left: Dose response curves (green) showing average moving and total embryos for N2 (a-b) and MY16 (c-d) strains in TPhP. Right: Dose response curves (green) showing average moving and total embryos for N2 (e-f) and MY16 (g-h) strains in piperazine. Red datapoints show the positive control 4 μM CH₃Hg. Slopes were fitted using nonlinear regression with a variable Hill slope. Dotted lines show the EC₁₀ and EC₅₀ values for each condition/strain.

FIGS. 29A-D show high-content multiparametric neurodegeneration phenotyping for DNT assessment. (a) Schematic of six dopaminergic neurons in dat-1::gfp C. elegans. (b) Fluorescence images of C. elegans exposed to a well-known neurotoxicant show increased dendritic degeneration phenotypes compared to vehicle control. (c) Multi-parametric degeneration percentages for different neurotoxicant concentrations. (d) Sub-lethal degeneration phenotypes scored.

FIG. 30 shows images from vivoChip®. The vivoChip®-2x enables rapid immobilization and high-resolution imaging of entire neuronal processes using air objectives (20×, 0.75NA) or oil objectives (up to 100×). Simultaneous immobilization of 40 animals side-by-side facilitates fast imaging and simple manual scoring of multi-parametric neuronal degeneration phenotypes using ImageJ software. High-resolution imaging of anesthetized animals (for complete immobilization) allows for the identification of even subtle neuronal degeneration phenotypes enabling investigation of the mechanisms of neuronal toxicity.

FIG. 31 shows images of BY200 (dat-1::gfp) animals immobilized in the vivoChip®-2x.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about y′”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “organism” as used herein refers to any organisms or microorganism, including bacteria, yeast, fungi, viruses, protists (protozoan, micro-algae), archaebacteria, plants and eukaryotes. Eukaryotes can be a single-celled eukaryotic cell. Bacteria include gram-positive and gram-negative bacteria. Organisms include, but are not limited to, bacteria, archaea, prokaryotes, eukaryotes, viruses, protozoa, mycoplasma, fungi, plants and nematodes. Different organisms can be different strains, different varieties, different species, different genera, different families, different orders, different classes, different phyla, and/or different kingdoms. Organisms can include genetically engineered organisms or genetically modified organisms. Any organism can be genetically modified.

### Methods

Disclosed herein is a method of analyzing internal components of an organism, the method comprising a) taking an image of internal components of the organism, while said internal components are contained in vivo, and b) analyzing the internal components. In one embodiment, this invention provides a fast method to analyze toxicity endpoints, specifically for developmental and reproductive toxicity (DART), in organisms. As can be seen in the examples, one embodiment includes imaging C. elegans hermaphrodites using brightfield microscopy, confocal microscopy, fluorescence microscopy, non-linear microscopy, structural-illumination microscopy, or polarized microscopy images.

As can be seen in the examples, the assay enables users to (1) feed age-synchronized populations (such as C. elegans being fed with bacteria) in various substrates, including but not limited to single-well or multi-well plate format, (2) co-culture with chemicals in an open or sealed environment while avoiding cross-contamination, (3) expose the organisms to a uniform and homogenous conditions (single chemical ingredient or their mixtures, for example, or to other various conditions), (4) monitor the growth parameters of the organisms at various intervals, constantly, or intermittently, (5) immobilize the organisms to image part or the entire body at high resolutions in 2 or 3 dimensions to capture different tissues and organs, or (6) acquire time-lapse images to track dynamical events within the organism's body.

Any organism can be used with the methods described herein. Examples of organisms include, but are not limited to, Caenorhabditis elegans, parasitic nematode, roundworm, C. briggsae, zebrafish, daphnia, planaria, and drosophila. Hermaphrodites, males, and females can all be used with the present methods.

By “in vivo” is meant that the organism is still completely in tact, or 60%, 70%, 80%, 90%, or 100% in tact. The organism can be alive or dead. The measurements can be used to determine the state of life, toxicity, state of health, or specific or general functions such as movement, excretion, eating, reproducing, etc. The organism can be partially or completely immobilized, or can be completely free moving. By “free moving” is meant that the organism can move at will about the container in which it is placed. It can also mean that the organism is constrained or restrained in some way, but still capable of moving all or part of its body. For example, the organism may be generally contained within a region, but be able to twitch, contract, expand, roll, undulate, etc. freely. Alternatively, the organism may be able to swim, walk, crawl, or fly within the space in which it is contained. When the organism is immobilized, it can be completely immobilized and not capable of any movement at all, or can be partially immobilized (10%, 20%, 30%, 40%, 50%, 60%, 70%, 80%, 90%, or more) so that part of the body is capable of movement while other part or parts are not.

By “immobilized” is meant that an organism can be fully or partially restrained or contained. This can be done by a variety of means. For example, the organism can be held on 2 or more sides by the walls of a chamber to physically constrain the organism. The cross sectional area of the chamber may reduce in a direction that the organism is pushed into in order to constrain it. Immobilization means the organism is substantially restrained from freely moving away from the imaging area, but its extremities may still be able to move. Immobilization may also be provided by surrounding the organism with a substance that can become solid or semisolid to restrict movement. This may be a substance which changes form upon temperature change, light, or addition of a second chemical. Alternatively, solid beads or particles may be packed around the organism between it and the chamber walls to reduce movement. The organism may also be immobilized through chemical anesthetic agents, changes in the oxygen or carbon dioxide concentrations in the environment, or a reduction in temperature to induce a hypothermic state. The immobilization can be performed by one or more of these methods at the same time.

The microfluidic device can comprise a plurality of features for the purposes of immobilizing multiple organisms simultaneously. For example, the device can comprise a plurality of individual isolated compartments to contain multiple separate populations of organisms.

As described herein, the organism or organisms can be in a conventional multi-well plate, agar plate, liquid culture, on solid media such as nematode growth media (NGM), gel, polymer microfluidic environment or microfluidic chip. The organism is at least partially immobilized using a physical confinement. This confinement can include, but is not limited to, gel, beads, or other means for immobilization. The microfluidic chip can be, for example, a multi-well plate.

When the imaging is used to determine how a certain substance (such as a chemical compound, composition, nucleic acid, protein, small molecule, etc.) or a set of conditions affects an organism, the substance or conditions can be applied to the organism before or during imaging of the organism. For example, when a substance is applied, this can be done in the gas phase or in the liquid phase. When applied in the gas phase, the substance can be applied in the chambers or headspace. When applied in the liquid phase, the substance can be applied in the culture media or absorbed onto the solid media.

The images can be captured either in their freely behaving phase or after immobilization. The organisms can be partially or completely immobilized. For example, freely moving organisms can be imaged (1) in the liquid culture, (2) on the solid media such as nematode growth media (NGM), gel, and polymer, or (3) inside a microfluidic environment. The images from a single organism or multiple organisms, freely moving in the culture, can be acquired using wide-field microscopes, confocal microscopes, fluorescence microscopes, non-linear microscopes, structural-illumination microscopes, microscopy with point-spread function engineering, computational microscopes, polarized microscopes, plate imagers, and flow-based imagers, for non-limiting example. To capture high-resolution blurred-free images of organisms, the organisms can be immobilized using (1) a conventional slide preparation to immobilize organisms (such as worms) using an anesthetic, agarose, gel, polymer, beads, or their combinations, (2) microfluidic technologies to immobilize organisms (such as worms), and (3) flow-based systems to capture the body dimensions of the organism in the presence of anesthetic.

The images can then be analyzed to characterize phenotypes. The whole-body images of the organism can be used to analyze body size parameters (such as body length, area, and volume). The change in their body dimensions, when compared with the control population (only solvent), indicates an adverse health effect due to the presence of a test chemical (single ingredient or their mixtures). The images can be used to analyze different organs (such as the pharynx, germline, uterus, intestine, vulva, muscles, etc.) in the organism's body. The organ-based phenotypes, such as organ shape, size, and separation between different organs, can detect organ toxicity at the whole-organism level. High-resolution images can provide information about inter-organ information such as embryos, pharyngeal lumen, cuticle integrity, muscle health, etc.). The time-lapse images of the body can be used to identify moving objects related to whole body movements such as motility and behavior and feeding. The time-lapse images with sufficient resolution can also provide analysis of transient events such as pharyngeal pumping, embryo movement, ovulation, organ twitching, defecation motor activity, etc. Coupled with high-resolution imaging and time-lapse monitoring of the in-utero embryo can help to identify different development stages of the embryos (such as early-stage, folded-stage, late-stage, and lethal embryos) within the parent bodies (in the case of animals such as worms). The analysis of the total number of embryos and their development can be related to the reproductive toxicity in the whole organism. In addition, the time-lapse images can identify subtle movement patterns in the late-stage embryos related to the developmental neurotoxicology effects, such as adverse effects on the motor neurons associated with the neuromuscular junction activities.

As proof of principle, the assay has been used to treat age-synchronized C. elegans and treat them with chemicals at different concentrations starting the larval 1 (L1) stage. This is described in more detail in the Examples. Specifically, the animals are cultured in liquid with bacteria and a chemical for 72 hours. The worm cultures are intermittently imaged directly from the plate and immobilized in a microfluidic technology to capture high-resolution images at 72 hours when the animals are reproductively active as day 1 (D1) adults. The subtle and sub-lethal phenotypic analysis of C. elegans allows us to assess the adverse effects on the parent's development and reproduction. Chemical toxicity can also be evaluated using fluorescent reporters' fluorescence imaging, demonstrating certain toxicology events such as organ health, organ development, aneuploidy, germline death, DNA methylation defects, defective sperms, etc. Although we showed DART in C. elegans hermaphrodites, it can be extended to males. A multiparametric analysis of subtle and sub-lethal phenotypes in C. elegans would help to improve the correlation in the concentration-dependent toxicity parameters in worms with other species, such as rodents, fish, and humans.

The organisms described herein can be cultured in solid, gel, and liquid media. The liquid-based assay, demonstrated in the Examples, uses 500 μL of culture media and contains the bacteria and the necessary ingredients for worm growth. The wholly sealed media can accommodate a wide range of chemicals (hydrophobic/hydrophilic, volatile, aromatic, etc.) and provide uniform exposure to the organisms growing for a specific amount of time before they are observed for phenotypic analysis. The assay can mimic oral exposures in mammals facilitated by ingesting the chemicals in various organisms. The use of sensitive strains such as a cuticle mutant in worms can uptake chemicals through the cuticle (in addition to the ingestion) to increase the local chemical dose at the worm tissues. Such versatile assays can be exploited for a wide range of substances including those that are insoluble in water (such as fragrance molecules and several (unknown or variable composition, and complex reaction products and biological materials also called as UVCBs). A simple culture protocol, performed by less trained scientists, using a large number of worms per population can provide phenotypic scores for DART-related endpoints with high-statistical power in a short time compared to the expensive toxicology testing using large animals.

One benefit of the present invention is that it allows one to analyze the embryos inside the parent's body before they are laid out. In addition, the parent development arising from acute or chronic exposures directly from the environment in which they are growing can be analyzed. The toxicology results from in-utero embryos (inside the parent's uteri) arise from exposure to the chemicals only through the germline availability and not direct exposure in the environment. Since the eggshell is very robust in C. elegans, for example, exposure can be determined by the chemicals inside each oocyte before the hard eggshell barrier is formed (post-fertilization processes).

The present assay enables a high-throughput approach with image-based whole-body screening that enables high-resolution phenotypic analysis. This assay allows label-free organ toxicity in the wild-type C. elegans strains (N2 and other wild isolates with diverse genetic backgrounds) without any genetic manipulations. Specialized strains, such as cuticle mutants and strains with fluorescent reporters, can also be used to identify subtle DART phenotypes or biochemical events associated with the toxicology mode-of-action (MOA) studies. The neuronal activity related to feeding, motility, and late-stage larvae development (obtained from the specific movement such as the twitching and rolling of worm larvae inside the eggshell) can inform about the developmental neurotoxicity of parents and new progenies. Such activities can tell about the adverse effects of chemical treatment on neuromuscular junctions and neuronal development.

We have ensured that the present invention, which allows for sealing the organism, provides sufficient media for chemical exposure, bacteria density for worm development (for example), and the head space for volatile chemicals. The sealed animals are only accessed intermittently to capture optical images to characterize their growth rates and, at the end of the study to assess the parents and the embryos (only present inside the parent's body) for DART-related parameters.

The present-day toxicology testing with large animal models relies on epical endpoints. The tests involving large animals are expensive and time-consuming. The present invention provides a solution to identify toxicology endpoints with sub-lethal and subtle phenotypic analysis in organisms, such as an invertebrate animal model. The study utilizes the soil-dwelling nematode C. elegans, with several advantages such as an intact reproductive system, whole organism physiology, and well-characterized toxicology pathways. The model can serve as an alternative to animal testing and complement other approaches such as in silico methods, in vitro cellular models, and other small animal models. Among other advantages, C. elegans has a transparent body, a short lifespan, and a natural microbiome, making it suitable for rapid screening of chemicals.

Many worms can be generated (each hermaphrodite producing ~300 progenies) and grown in liquid media to allow uniform chemical exposures for acute or chronic exposures. Using DART-related phenotypes in C. elegans from parents and their progenies, one can analyze multiple parameters very quickly compared to the conventional methods. One of the assays uses 72-hour chemical exposure of adults and identifies in-utero embryo developmental parameters (number of embryos and their developmental stages).

Disclosed herein is a robust assay to collect phenotypic information from many animals per treatment condition and provide endpoints with high statistical power. With such high-statistical power and multiparametric analysis, high predictability power becomes available in DART assessment. The sensitivity of the assays is further increased by analyzing the phenotypes in a panel of wild isolates with diverse genetic backgrounds to capture the variability.

Current-day C. elegans-based assays use intoxicated adults to produce, lay, and hatch embryos from parents to assess developmental toxicity. The assay depends on egg-laying behavior to lay ~300 embryos within up to 5 days. The embryos then hatch outside the parent's body and are counted to assess hatching rates and the total number of progenies. In this assay, the parent's health can be characterized by measuring its developmental features (body size, organ-level toxicity, and behavior) at the end of the culture time. The reproductive health is assessed using embryo imaging and identifying their development inside the parent's uterus. The study mitigates difficulties associated with long worm cultures, laborious protocols, and saves experimental time.

Since egg laying is controlled by a particular neuromuscular activity, adverse effects on the neuromuscular junction can alter the DART phenotypes, demonstrating off-target toxicity (egg-laying defects and the bag-of-worms phenotype). The present assay, directly measuring the embryos within the parents, mitigates the off-target effects while providing the health of the embryo development and the parent's reproductive health.

The newly hatched larvae, once outside the parent's body and the eggshell, are exposed to the chemicals directly. Young larvae have very fragile cuticles and are susceptible to chemicals. Depending on the duration of the chemical exposure and the concentrations, larvae can show severe adverse effects and produce heterogenous toxicology phenotypes. In contrast, in this assay, the embryos are only exposed to toxic chemicals through the parents, presumably those inside the germline. The embryonic phenotypes captured in this assay, while they are inside the eggshells, are more robust and not influenced by chemicals present in the culture environment. This is analogous to chemical exposure in the human population.

The current study analyzed up to 12 C. elegans strains and used the time-lapse brightfield or fluorescence data to analyze the parent and embryonic development phenotypes. The assay can be extended to other organism using specific fluorescent reporters to identify parental defects (such as defects in the muscle, other organs, neurons, etc.), embryo defects (such as aneuploidy, chromatin organization, and packaging, sperm count, fertilization process, ovulation, etc.). One can acquire time-lapse data with more prolonged periods and/or fast intervals to capture events occurring in organisms, such as ovulation, sperm motility, pharynx pumping, cell division inside the eggshell, embryo twitching pattern, etc. The study can be extended to animals at different stages of adulthood (linked with young vs. aging parents), exposed to chemicals for different periods (chronic, sub-chronic, acute), and co-culture with substances to avoid toxicity (chemicals to prevent adverse outcomes for therapeutics).

The assay can be used for mode-of-action studies involving RNAi and genetic markers for toxicology pathways (such as ROS formation, stress reporters, and degeneration).

The assay can study fundamental questions in reproductive biology and screen a gene or its product involved in reproduction. The C. elegans germline and uterus have been used to model human diseases such as cancer, aging, and regeneration, which can be screened in the assay.

As mentioned above, a variety of different internal components can be imaged using the methods described herein. For example, the component can be a reproductive/germline component. The reproductive component can be a gamete, embryo, stem cell, or chromosome, for example. The gamete can be an egg, oocyte, or sperm. The reproductive component is a fertilized egg. The reproductive component can be an embryo. The internal component can also be an organ, such as those from digestive, reproductive, nervous, sensory, muscle, or excretory system, specifically such as pharynx or intestine.

Various parameters can be assessed by imaging the organism. These parameters can then be used to compile a score, which can be used to determine a variety of metrics such as toxicity. More than one phenotypic character can be scored at a time, so that a “multiplex” assay is created for measuring different parameters simultaneously or sequentially. A score can be compiled over time, and can be compared to a control or to different time points of the same organism, such as before exposure to a substance. This can be used to determine functionality of internal components, and various parameters can be measured. Different organisms can be assayed simultaneously, as well as more than one of the same organism.

Machine learning, or automated machine vision, can be used to classify. By way of example, the machine learning can be a deep learning neural network that can classify phenotypic assay data, such as high-dimensional images. To train the deep learning neural network, each of the high-dimensional images are labeled with a clinical phenotype and the deep learning neural network is trained to improve its clinical phenotype prediction. In various embodiments, a loss function is employed, the loss representing a penalty that is the difference between the prediction of the deep learning neural network and the clinical phenotype label of each image. Therefore, the loss can be back-propagated and the weights and biases of the neural network are adjusted to minimize the loss. In various embodiments, the deep learning neural network can incorporate any of the leading deep learning platforms such as TensorFlow, Keras, Pytorch, Torch, Theano and Caffe. Therefore, the trained machine learning model includes a relationship that aligns the high-dimensional data of the phenotypic assay data (e.g., an image) to the lower dimensional output (e.g., predicted clinical phenotype).

Various metrics of the organism can be analyzed, including but not limited to n volume, size, and/or relative positions of whole body or individual organs, organ contents, and/or their texture are evaluated. Dynamical events, such as movement, of the internal component can be measured. Growth/progression of the reproductive/germline component can be measured as a function of time. Malformation or slowed progress in formation of the reproductive/germline component can also measured. The images can be analyzed to determine phenotypes relevant to developmental and reproductive toxicity. The organism can be genetically engineered, for example.

The images can be analyzed to determine the phenotype relevant to neurotoxicity. The term neurotoxicity refers to damage to the brain or peripheral nervous system caused by exposure to substances. These toxins can alter the activity of the nervous system in ways that can disrupt or kill nerve cells. These neurons can be analyzed, and can belong to a specific neurotransmitter. Examples include, but are not limited to, GABAergic, Dopaminergic, serotonergic, or Cholinergic neurons.

The internal component can be the intestine. Intestinal permeability of the organism can be assessed. The method of assessing intestinal permeability can be measuring the uptake of a colored or fluorescent dye from the intestinal lumen.

The internal component can also be muscle. The muscle can express a fluorescent reporter gene. The fluorescent reporter gene can mark protein aggregation. It can also mark oxidative stress.

The internal components can also be lipid storage structures. The lipid storage structures can be stained with a dye marking specific lipid subtypes.

Also disclosed is a method of determining toxicity or efficacy of a substance, the method comprising: exposing a organism to one or more substances of interest; taking one or more images of one or more internal components of the organism, analyzing internal components of the organism from the images to determine the effects of the substance on the organism, and using the results of the analysis to determine the toxicity or efficacy of the one or more substances.

### EXAMPLES

### Example 1: a High-Throughput C. elegans-Based NAMs to Study Developmental and Reproductive Toxicity (DART)-Related Endpoints

Developmental and reproductive toxicity (DART) is a vital part of product safety testing for chemicals, consumer products, and drugs. Its purpose is to identify potentially hazardous ingredients that can cause abnormalities in an organism's development or reproductive function. Most of this testing is currently carried out in animal models such as mice and rats for human toxicity risk assessments, or other model vertebrates such as zebrafish for ecotoxicology. However, with the goal of reducing animal usage, new legislation from the US, EU, and other countries are taking actions to limit the use of these models. Additionally, the cost of assays using mammalian models or fish is large due to their complex housing requirements and size, assays are slow because of the organism's long life cycles and may have low statistical power as the study population sizes are limited by the high costs. Despite their high genetic and physiological similarities to humans, rat data is only ~50% predictive of human toxicity. With over 350,000 chemicals registered today and requiring new or updated testing [1], there is a need for New Alternative Methods (NAMs) to replace current methods.

NAMs can use in silico predictions, in vitro methods [2], or invertebrate alternative model organisms not subject to welfare regulations such as C. elegans, zebrafish or medaka larvae [3,4], brine shrimp [5], and Daphnia [6]. However, no method is capable of completely recapitulating mammalian biology. In vitro methods still require complex and expensive culture protocols and cannot replace DART methods that require a complete life cycle and reproductive system. Fish larvae assays have met with wide success but still require hard to maintain aquariums, have relatively long-life cycles, and are considered animals subject to welfare regulation once they pass the larval stage. C. elegans is a nematode small model organism that has been widely used in scientific studies since the 1960's, including many in toxicology. It can be grown quickly and cheaply in large numbers using relatively simple techniques and apparatus. Thanks to its small size and simple, transparent body structure, it is highly amenable to imaging-based methods of analysis. The C. elegans genome has been sequenced and shown to have many of the same metabolic and signaling pathways as mammals, and up to 83% homology with the human proteome [7]. It has complete neuromuscular, reproductive, and digestive systems with conserved mechanisms to humans making it amenable for Adverse Outcome Pathway toxicology methods, and also contains at least 76 cytochrome P450 genes [8] and has all 3 phases of xenobiotic metabolism [9] and a functional microbiome. These advantages make it highly suited to toxicology testing, especially high-throughput assays at relatively low costs [10,11].

Use of C. elegans has been limited in toxicology before due to the difficulty in examining large enough populations to get statistical significance over a wide dose range for anything other than a few phenotypes such as body length and brood size. However the studies that have been performed show good concordance between C. elegans and mammalian data, with around 53% balanced accuracies for DART parameters between C. elegans and mammals [12]. In certain chemical spaces, for example non-acidic chemicals, C. elegans shows 0.885 concordance with rat predicted toxicities, equaling the 0.879 concordance between that of the mouse and rat [13].

A study was conducted to develop an imaging-based assay to quantify DART toxicity using C. elegans with a high-throughput microfluidics platform that is amenable to automation and can be scaled up to perform large numbers of assays. The assay can be performed in 3 days from initial worm plating and treatment to imaging endpoints. The assay readouts are purely based on brightfield image analysis, can be performed with any C. elegans strain, including a transgenic line or the wild isolates with a diverse genetic background. The study found that 4D imaging of the entire germline allowed identification and quantification of the developmental stages of every embryo in the parent, which was a more sensitive measure of toxicity than body size alone. The in-utero analysis of embryo development is unaffected by the off-target effects such as neuromuscular defects on the egg-laying circuits. The study showed that the assay protocol, carried over extended time periods of months, by several personnel, with multiple Dauer stocks, and with multiple thawed batches of strains, is highly robust and produces repeatable results for multiple phenotypes. The study tested the assay of a commonly used agricultural chemical of concern to environmental toxicology: propiconazole, which has published DART toxicity data in other species, including Daphnia, rodents, amphibians, and fish.

### Materials and Methods

Strains and maintenance: The study used the N2 wild type (The Caenorhabditis Natural Diversity Resource, CaeNDR) for all experiments except for the initial vivoChip-2× pilot study, where the study used N2 wild types from the Caenorhabditis Genetics Center (CGC). C. elegans were maintained using the standard methods on nematode growth media (NGM) plates seeded with HB101 Escherichia coli bacteria and maintained at 20° C. [14]. Frozen stocks of strains were also maintained at −80° C. and thawed each year to generate a new set of Dauer plates. Sealed Dauer plates for each strain were stored at 16° C. and used to start a fresh maintenance culture every 3 months by chunking to avoid genetic drift. Worms were cultured for >5 generations before using them for assays in a temperature and humidity-controlled environment.

Food preparation: HB101 was streaked on a Luria-Bertani (LB) agar plate with streptomycin (antibiotic) and incubated overnight at 37° C. to generate single colonies. One colony was picked and used to inoculate an overnight 5 mL starter culture in LB broth and antibiotic. 200 μl of this starter culture was added to 200 mL LB broth/streptomycin in a 1000 mL baffled flask and incubated for 18 h with 180 rotations per min (rpm) shaking to reach the end of the log growth phase. OD₆₀₀ was measured using an Implen DiluPhotometer, and the culture was stored at 4° C. Immediately before worm culture, a sufficient volume of the bacterial culture was centrifuged to generate the required volume of OD₆₀₀^{=3.0} food suspension. The LB supernatant was removed and the pellet was resuspended in S media. This density was optimal for growing 80-100 larval 1 (L1) to the day 1 (D1) adult stage in a 500 μl culture volume with food remaining after 72 hrs, but not so much residual food particles as to interfere with imaging or chip operation.

Chemicals: Methylmercury (II) hydroxide (CH₃Hg, Alfa Aesar, CAS #1184-57-2) and 5-Fluoro-2′-deoxyuridinc (FUdR, TCI Chemical, CAS #50-91-9) were dissolved in ultrapure water as master stocks at 200× highest treatment concentration. Propiconazole (Sigma Aldrich, CAS #60207-90-1) was dissolved in dimethylsulfoxide (Sigma Aldrich, CAS #67-68-5) as master stocks at 500× highest treatment concentration. The master stocks were transferred into 0.2 ml tubes as single-use aliquots and kept at −80° C. until needed. The 1 μM dose of CH₃Hg was prepared by diluting the stock in ultrapure water prior to the treatment. For the dose-response assays, propiconazole was diluted immediately prior to treatment by serial dilution in DMSO to 500× working dilutions as needed, so that the final DMSO concentration in each well would be 0.2%. DMSO was also added to both the solvent control wells and the CH₃Hg control wells to 0.2%.

Worm synchronization and large-scale culture: To prepare large numbers of synchronized L1 stage larvae, P₀ larval 4 (L4) worms were transferred to NGM plates and grown for 96 hrs till the F₁ generation reached maturity, removing the P₀ adults after 48 hrs. D1 animals were treated with an alkaline sodium hypochlorite solution with periods of slow and high-speed shaking on a speed-controllable vortex to fragment their bodies and release the embryos before the bleaching solution was neutralized with 5 washes of M9. The eggs were allowed to develop and hatch in the M9 buffer in a rotating glass conical tube for 24 hrs. Hatched synchronized L1s were filtered through a 20 μm cell filter to remove unhatched eggs and debris, counted the number of total larvae, and adjusted the volume to make a final suspension of L1 larvae at a density of ~100 L1s/20 μl.

Chemical treatment in liquid culture: The age-synchronized L1 suspension (~100 larvae in 20 μl volumes) was placed in a standard 24-well plate with 480 μl HB101 (OD₆₀₀^{=3.0}) suspension in S media. 2.5 μl of chemicals dissolved in H₂O, or 1 μL of the chemicals in DMSO were added to the designated wells, keeping solvent concentration constant across all wells. Vehicle control wells received the same volume of each solvent (H₂O or 0.2% DMSO). The 24-well culture plates were sealed with airtight film (Thermo Scientific, 232702) to prevent evaporation or cross-contamination and cultured at 20° C. for 72 hrs. Timelapse imaging (5 frames over 5 s with a 2×, 0.08 NA objective) was performed every 24 hrs to monitor development and ensure the control wells developed to D1 adult stage according to the acceptance criteria. The plates were gently agitated after each imaging session to homogenize the well contents, including the HB101 concentration, shed cuticles from molting, and the intestinal discharge from the defecation cycles over 24 hours.

vivoChip-24x design and its use: Microfluidic-based C. elegans immobilization chips made of Cyclic olefin copolymer (COC) were used for the experiments in this study. COC was chosen as the material for the microfluidic parts due to its low chemical absorptivity and its wide optical transmission spectrum. These chips were designed with 24 separate on-chip wells (vivoChip-24x) positioned in a standard 96-well format of 9 mm well spacing. Each well is connected at its bottom surface to the input region of a microfluidic channel array including 40 parallel tapering channels, as described previously [15]. The tapered channel geometry of the 960 channels in each chip orient and immobilize ~40 C. elegans simultaneously from 24 different worm populations for high-content imaging.

The seal of the 24-well culture plates was removed, and all the worms were pipetted from the culture plate to the corresponding wells of the vivoChip-24x using a multichannel pipette.

The chip was placed in a custom gasket system based on a previously described system [15] and sealed for fluid connections. The gasket and chip were placed on the microscope stage for imaging. Flow was initiated with pressure cycles across the wells controlled with the vivoCube+microfluidic control system for 10 cycles of on/off pressure. Immobilization of C. elegans in all 960 channels in the chip took ~3 minutes.

Automated imaging platform: The vivoScreen system was built around a customized large field of view inverted microscope (IX73, Evident), a precision xyz stage (MS2000, Applied Scientific Instrumentation), sensitive scientific CMOS camera (IRIS-15, Teledyne), and bright light sources (TLED, Shutter Instrument). The large field of view setup could acquire the entire chip area with a 2×, 0.08 NA objective, and 8 channels at 10×, 0.4 NA objectives requiring 5 FOVs to cover all 40 channels. The channel positions were mapped in x and y based on known channel positions from an initial positioning at well A01, plus the best-focus plane finding of cross-shaped markers adjacent to each trap for the z portion. The map enabled rapid imaging of all channels in the chip at 2×, then at 10×. A worm-finding AI algorithm was used to position the FOV at the optimal channel position to capture most worm body areas at 10× using the 2× images. The study included development of a custom software (vivoImager) to control the microscope, stage, camera, illumination source, peripheral optics, and store the ~180 GB of collected 4D hyperstack images of all the animals in the chip in an attached server with experimental metadata files.

Image processing and phenotypic scoring: The images of individual channels were cropped based on the fiduciary markers on each well and the relative distances between the parallel channels. The cropped hyperstack images for all 960 channels were stored on the local server for multiparametric analysis.

A deep learning-based method was used for the automated detection of the C. elegans body. In brief, an image slice with optimal focus from the image stack was identified by calculating the variance of the Laplace-transformed image. An improved U-Net architecture, including encoder layers, a bottleneck layer, decoder layers, and a classification head at the bottleneck was used. The final trained model was used to get the body phenotype (no worm, partial worm, or full worm body) and the predicted segmentation mask (2D pixels associated with a worm body). The predicted mask was used to calculate body length, area, and volume for each worm.

Embryonic phenotype scoring was performed by 5 different trained scorers using the custom graphical user interface (GUI) based image display and annotation software (vivoAnalyzer). The software loads each cropped individual channel and presents it to the user with the ability to animate through the timelapse frames and move up and down z-planes. Markers could be placed on each embryo in the image and assigned a particular phenotype (moving stage embryo, folded stage embryo, or early-stage embryo). Scoring data was stored in an SQLite database and exported for statistical analysis. The treatment condition of the image was not displayed to the scorers.

Statistical analysis: Assay power estimates were calculated using the average total embryo measurement and average standard deviation for H₂O control populations from the vivoChip-2× pilot experiments with 2 samples of n=40 at α=0.05, and desired minimum detectable effect sizes of 5-50% of the control mean.

For DART data analysis, data was first filtered to remove all measurements from worms with body sizes or embryo numbers that significantly deviated from the median. Closer inspection of these rejected samples found them to be mostly pre-adult worms that had not developed to the expected stage by 72 hrs, or in rare cases dead/damaged animals. Tukey fences (1.5*the interquartile range (IQR)) were set for body length. Any animals with measurements that lay outside these fences were removed from analysis. The filtering process was then repeated on the remaining set using total embryo number as the criteria.

To estimate the number of worms per device and the number of experimental repeats required to obtain results with an acceptable low coefficient of variation (CV), the study randomly sampled data 1,000 times from 6 experiments performed on different days, and from one of 6 replicate wells in each as follows: (1) The data was filtered to remove biological outlier animals as above. (2) k wells were chosen (k=2 to k=6) at random from the 36 total wells for each condition (without sampling more than 1 well per experiment) to simulate a set of k experimental repeats, each with a single well per condition. (3) As an additional step to determine the effects of the number of worms analyzed per well, j worms (j=8 to j=40) were chosen at random from each well (4) Averages scores of all worms were calculated for each well for a given phenotype using j worms and k wells. (5) The CV and 95% CI of the k well averages were calculated for each sample. Data filtering and sampling was conducted using Python scripts.

Dose response curves and EC₁₀ or EC₅₀ values were fitted from datasets with a 4-parameter, variable slope Hill function using the “Find ECanything” nonlinear fit function of Graphpad Prism, with the slope bottom constrained to 0 and the 95% confidence interval bands calculated and plotted.

### Results

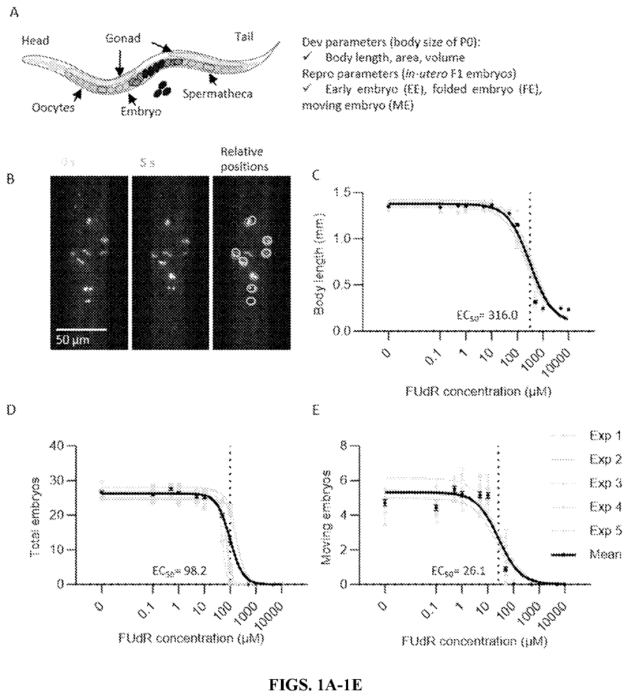

Development of a robust C. elegans assay to study in vivo developmental and reproductive toxicity (DART)-related endpoints: C. elegans has previously been used as a model for testing the developmental toxicity of various substances. However, previous studies have relied on post-embryonic development and fecundity, or single developmental phenotypes such as body length. Some also required transgenic reporter lines. This study hypothesized that high-resolution, bright-field imaging could be used to quantify multiple sub-lethal developmental and reproductive phenotypes in any strain, to gain a more accurate and sensitive picture of a substance's DART effects. The vivoChip microfluidic devices previously developed [15,16] allow high-throughput worm immobilization and 3D imaging of the C. elegans germline at resolutions sufficient to determine the developmental staging of in utero embryos [17] (FIG. 1A). To add additional sensitivity, the neuromuscular movement of individual embryos inside the immobilized parent can be monitored using timelapse imaging which correlates with advanced development [18] (FIG. 1B). The length and cross-sectional body area of trapped worms can be measured, and because the exact height and width of the microfluidic channels are known, highly accurate determinations of body volume, the C. elegans analog of body weight determinations used in standard mammalian toxicity tests, can also be performed [19]. Here this study proposes a multiparametric assay using C. elegans to quantify DART endpoints with high sensitivity, accuracy, and statistical power.

For the initial testing of this hypothesis, this study explored the response of wild-type N2 worms when treated various doses of 5-Fluoro-2′-deoxyuridine (FUdR), a drug commonly used to prevent the development of live offspring in C. elegans experimental cultures [20]. Age-synchronized N2 L1 larvae were plated with liquid media and food in a 24-well plate, treated with 0-10,000 μM FUdR, and cultured at 20° C. for 72 hrs. All the D1 adults were imaged in a small-scale vivoChip with a 10× objective at 1 fps frame rate for 10 s. The length of all worms in each concentration were manually measured by taking the distance from the head to the tail of each animal. The study counted the total number of embryos and the number of embryos visibly moving within the eggshells in each worm through the time stacks (FIGS. 1C-1E). It was found that FUdR caused a significant reduction in the number of total embryos (EC₅₀=98 μM) and body length (EC₅₀=316 μM). The moving embryo parameter (EC₅₀=26 μM, FIG. 1D) was more sensitive than the body length or total embryo parameters. These results indicated that it was possible to detect developmental and reproductive abnormalities using 4D high-resolution imaging of C. elegans and their in-utero offspring and that developmental staging of in-utero embryos is a more sensitive indication of the lowest adverse effect concentration (LOAEC) of a chemical than body length alone.

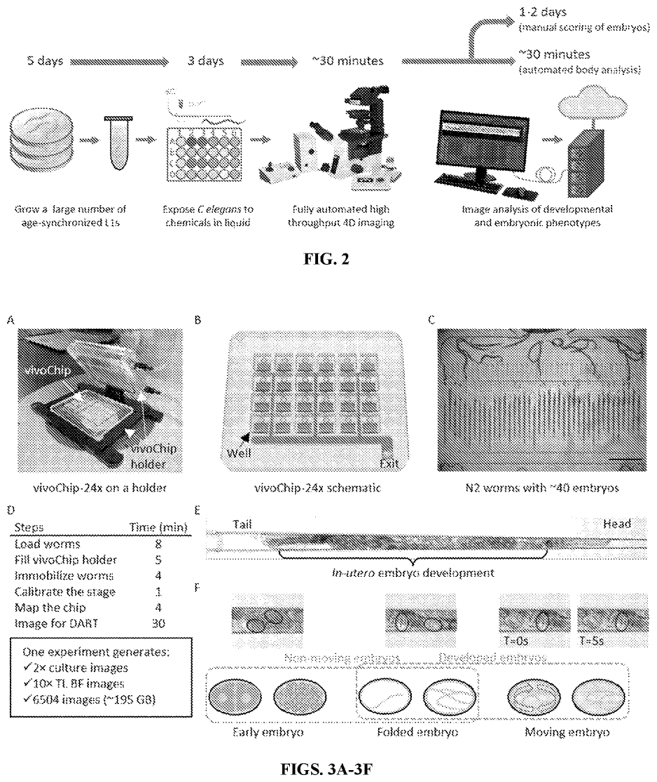

High-throughput assay using vivoChip-24x device: Based on these observations, the study developed a large-scale C. elegans DART assay to allow rapid high-throughput screening of chemicals (FIG. 2). The study based the assay around the innovative vivoChip microfluidics platform thanks to its ability to rapidly immobilize large numbers of animals for high-resolution imaging. The study estimated imaging durations for various chip capacities and concluded that a 24-well device (capable of trapping up to 24× 40=960 animals) was optimal considering a relatively short <30-minute imaging duration of all trapped worms in 24 wells and 1:1 compatibility with 24-well microplates, used for worm culturing and treatment.

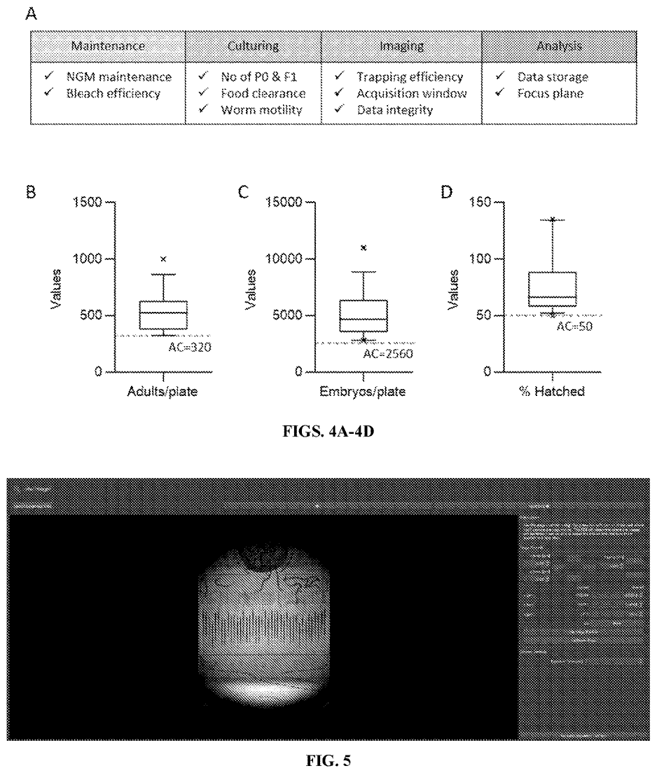

The vivoChip-24x includes three layers, a transparent plastic top layer containing 24 rectangular wells, a microfluidic layer made of COC material, and an ultra-thin 200 μm COC bottom sheet for enabling high-resolution imaging. The wells have capacity of 250 μl liquid volume for worm loading and directly connect below with the microfluidic layer having 40 trapping channels per each well. A pressurized gasket system seals the vivoChip-24x device and applies pressure necessary for loading and immobilization similar to previously published designs (FIGS. 3A-3B).

C. elegans maintenance and culturing for a repeatable assay: To make the assay of value and allow predictive toxicology to be performed, the results obtained needed to be precise and repeatable. This required minimizing the biological sources of experimental variability as much as possible. In short, each population of worms that entered a chip after being cultured under the same treatment conditions needed to be biologically identical. The study developed and followed standard operating procedures (SOPs) covering certified chemicals, calibrated instruments, well-characterized strains, a sterile environment, and well-trained personnel for the assays. Observations were recorded during intermediate steps during the assays, quantifiable data was acquired whenever possible, and all the data was safely stored in concordance with previously published design considerations and criteria for an ideal C. elegans toxicology assay (FIG. 4A) [21]. This protocol covered producing synchronized batches of healthy L1 larvae by sodium hypochlorite bleaching and growth arrest, addition of toxicants and solvents, 72 hr liquid culture in S media with HB101 food, and transfer to the vivoChip-24x for imaging. To ensure batch-to-batch quality, the study checked key steps of the protocol against a set of acceptance criteria, and only proceeded with analyzing experiments that passed (FIGS. 4B-4D).

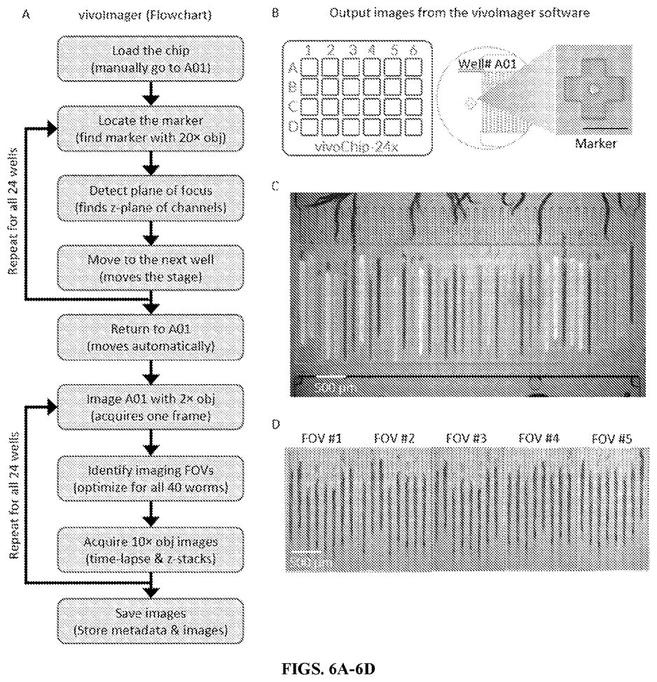

Automated imaging platform for high-volume image acquisition: To acquire the imaging data, the study developed fully automated image acquisition software (vivoImager) that controls a motorized inverted wide-field microscope and its peripherals including stage, camera, objectives and illumination, to capture 10×, 0.4NA 4D hyperstacks of all worms in their best focal planes (FIG. 5, FIG. 6A). Each device in the vivoChip-24x incorporates a cross-shaped feature on the microfluidic channel layer to allow the software to automatically identify the best focal plane for each device using an edge-detection algorithm (FIG. 6B). Initially, the user manually positions the microscope stage over the cross at the first well (well A01). Using a built-in chip map, the software moves the stage to the known cross positions at each well and finds the best focal plane for each FOV (plane differences are mainly due to curvature of the COC substrate under chip pressurization). While the relative channel locations are known from the chip map, the position of the trapped worms within 40 parallel and 3 mm long channels is unknown, and so the best position to center the 1.3 mm×2.2 mm FOV to capture the entire body of as many worms as possible needs to be found (FIG. 3C). First, a low-magnification image of the entire chip is captured using the 2×, 0.08 NA objective. Then all the worms immobilized in the 40 parallel channels are detected using a trained Detectron2 object detection machine learning (ML) model and the relative stage positions calculated of the 5 FOVs needed to image the worms at 10×, 0.4NA (8×150 μm spaced channels can be fitted in each 1.3 mm wide FOV) (FIG. 6C). It then automatically switches to the 10× objective and uses the 3-axes high-precision translational stages to capture 250 images (5 FOVs per well ×10 z-stack planes each with 6 μm step size per second×5 seconds) per well (FIG. 6D). One full vivoChip-24x experiment generates 6,504 images (~195 GB), including the culture plate images (FIG. 3D). Raw images and metadata (timestamps, xyz coordinates, image capture and illumination parameters, treatment conditions etc.) are sent to a high-capacity network attached storage (NAS) server immediately after each experiment for preprocessing (cropping of individual worms to separate images) and later analysis.

The optical resolutions and time-lapse lengths are sufficient to identify embryo phenotypes indicative of developmental stage. Each embryo was phenotyped as one of three developmental categories based on morphology and movement: moving (late-stage developed embryos close to being laid, usually positioned in the part of the germline close to the vulva), folded (partially developed embryos that had undergone at least the first folding event of gastrulation), and early (embryos with no signs of development). Embryo phenotypes can also be grouped together into developed (moving plus folded, all embryos that have progressed past at least the 1-fold stage) and non-moving (early plus folded, embryos that have not reached advanced developmental stages with neuromuscular activity) (FIGS. 3E-3F).

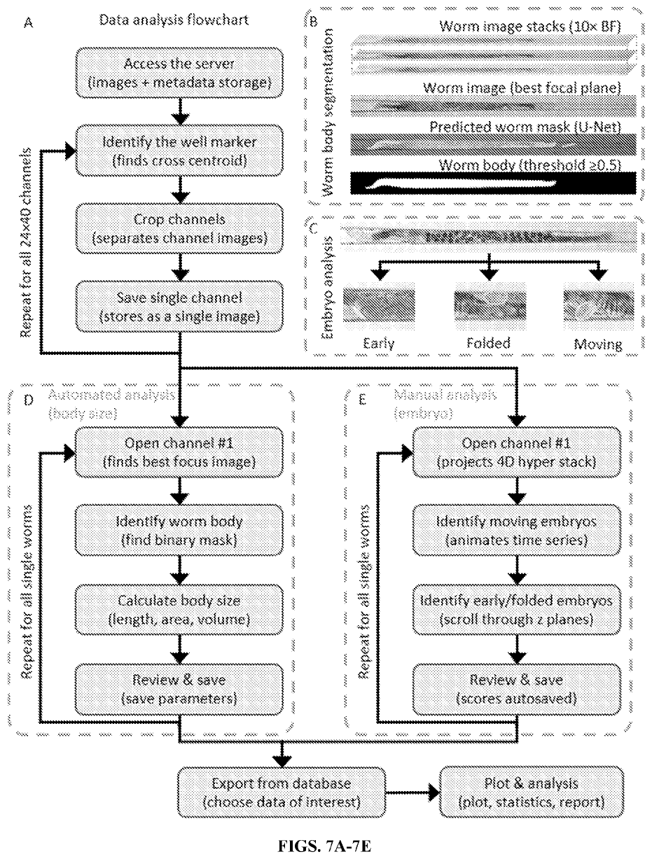

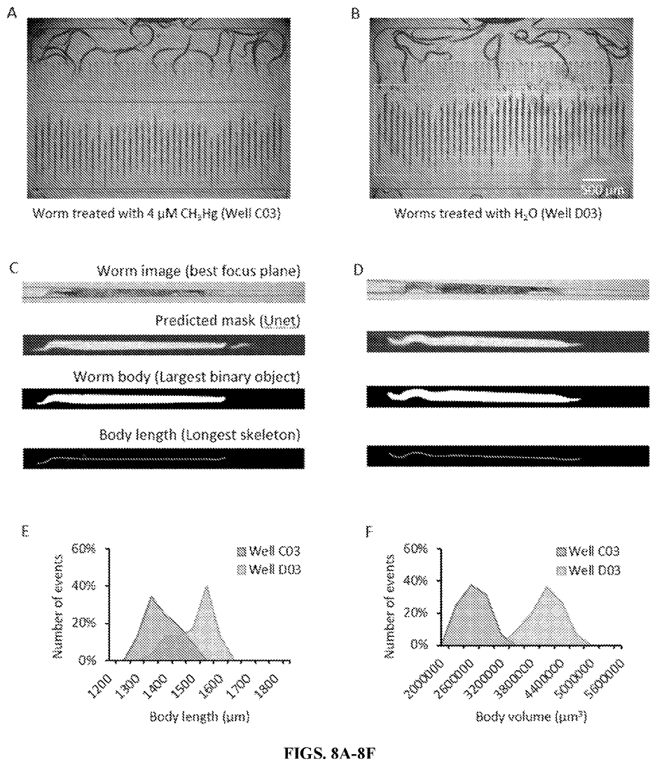

Image analysis pipeline for robust phenotyping: Imaging data is first processed by cropping out individual animals as separate 4D hyperstacks (FIG. 7A). Example images of the phenotypes analyzed are shown (FIGS. 7B-7C). Image analysis of the 4D hyperstack images involves two separate processes; (1) analyzing the worm body dimensions using a fully automated deep-learning-based ML platform (FIG. 7D) and (2) counting of embryonic phenotypes facilitated by GUI-based analysis software to streamline as much of the manual scoring process as possible (FIG. 7E). The study developed an in-house ML-based model by training it using 2,700 manually segmented body images. This model can detect the worm body boundary within the channels with ~97% accuracy (FIGS. 8A-8D). From the segmented pixels, body length can be calculated using the longest skeleton analysis and body area from the largest binary image (threshold ≥0.5) (FIG. 7D). Body volumes are then estimated by multiplying each pixel in the area mask by the known channel height at that location. The model detects each worm and calculates its body parameters from each worm population. Population differences between toxicated and untoxicated worms can be greater for the volume phenotype than the length phenotype (FIGS. 8E-8F).

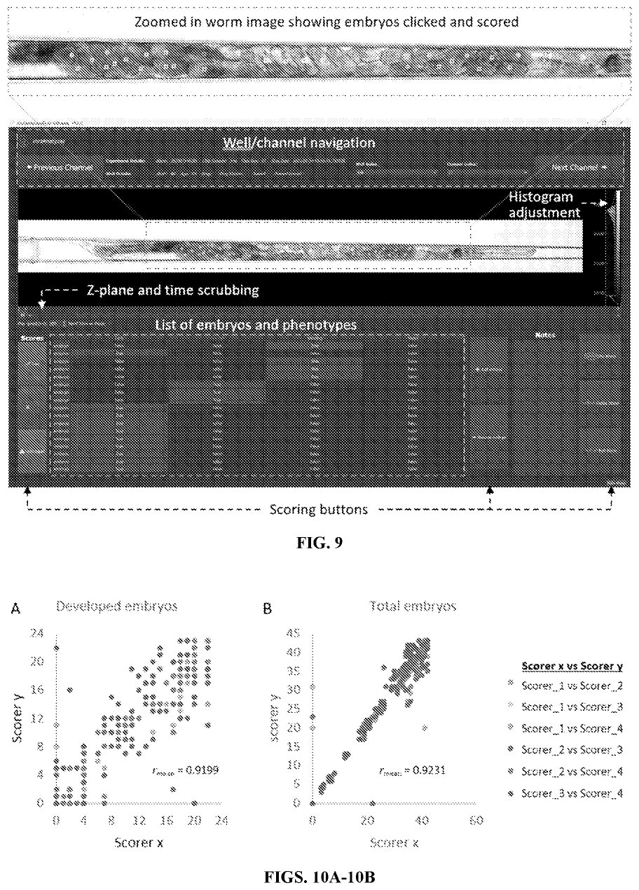

Embryo phenotyping is a daunting task since each vivoChip-24x can immobilize up to 960 adult C. elegans, which necessitates scoring ~30,000 in-utero embryos. Additionally, the intertwined morphology of the germline with the intestine and the presence of gut granules require 3D visualization of the worm uteri for robust phenotyping. To facilitate robust embryo phenotyping, the study developed GUI-based software to automatically display each cropped animal in turn, animate through the frames to show embryo motion, allow the user to zoom and scroll through the stack, click on the xyz centroid of each embryo, and assign them a phenotype (FIG. 9). Scoring was carried out by 5 users after their initial training on a sample dataset and validation of their scoring accuracy (FIGS. 10A-10B). The treatment conditions for worms are not displayed to the scorers to reduce scoring bias.

Repeatable DART results using the vivoChip-24-based assay: To show the value of the assay in predicting DART toxicity of substances with high statistical power, the study explored repeatability of predictions of toxicity levels across multiple tests of the same sample. For an assay to be capable of selectively replacing or augmenting, it needs to be scalable, reproduceable by different scientists using different batches of worms at any point in time, and resulting in repeatable data.

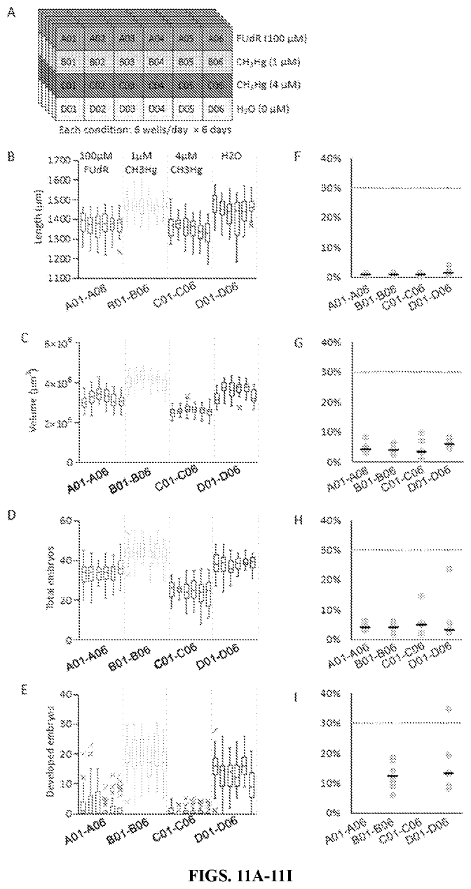

The study tested the repeatability of the DART assay by performing a series of 7 repeated set of experiments over 6 months using the same concentrations of two known toxicants FUdR (100 μM), (1 μM and 4 μM) methyl (II) mercury (CH₃Hg) [22], and a water solvent control in different batches of N2 worms (FIG. 11A). Two of these experiments used a new starter population and a Dauer batch of N2 expanded from the same frozen stock. One experiment was identified as not passing the acceptance criteria for development and was therefore rejected from the analysis. Each condition was replicated across 6 wells to identify well-to-well (technical) variability. This approach gave a total of 6 experimental repeats, containing 6 technical replicates each. The median trapping efficiency across all microfluidic devices in all chips was ~97.5% (39 of the 40 trapping channels of each microfluidic device had worms in them). In total, the study imaged and analyzed 5,117 worms and 179,529 embryos across all conditions.

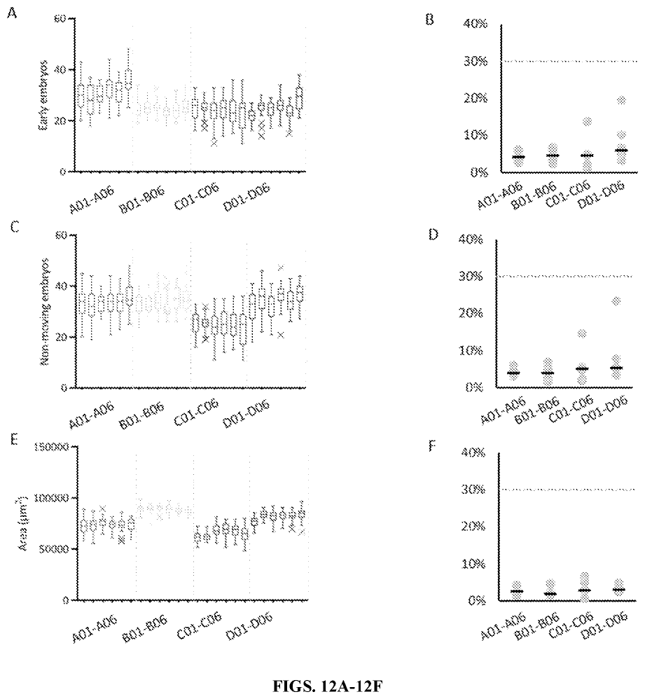

Raw data for each worm was loaded and scored for embryo phenotypes and body parameters using vivoAnalyzer and the ML model, respectively. The raw data was first filtered using Tukey filtering of worm length and then total embryos to remove biological outliers within each worm population (e.g. animals that were significantly below median D1 adult size or with no embryos in the H₂O control populations). Moving and folded scores were aggregated as developed embryos to reduce variability. The study obtained highly similar assay results in all the phenotypes studied from all 6 experiments, including the one performed months later using a different maintenance culture of N2 (FIGS. 11B-11E). The median coefficients of variation (CV) between replicate wells for H₂O control populations for the length (1.6%, FIG. 11F), volume (6.0%, FIG. 11G), total embryos (3.2%, FIG. 11H), developed embryos (13.3%, FIG. 11I), and early embryos (6.1%, FIGS. 12A-12B) were much below the 30% threshold, representing accepted values in a similar species in the OECD test guideline #222.

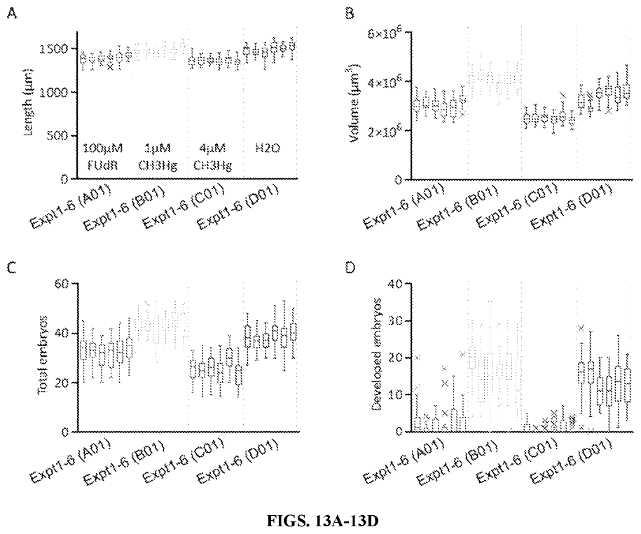

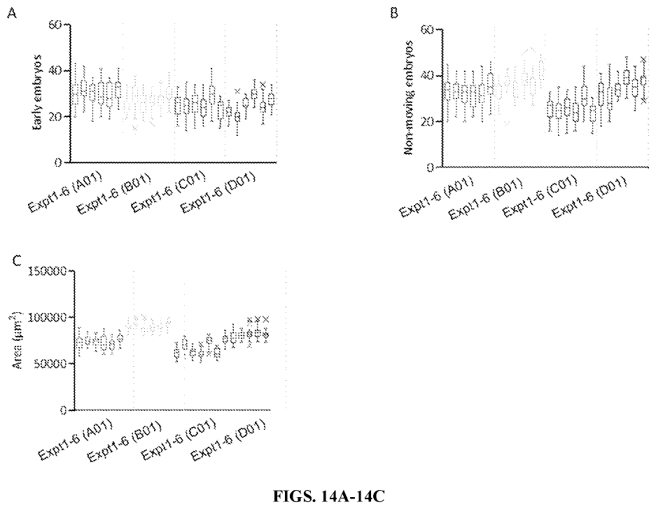

To identify the variation between technical repeats on the same day versus on multiple days, the study also analyzed the experiment-to-experiment repeatability by comparing corresponding wells from each experiment to each other e.g., well B01 from all experiments. The data points for each worm within corresponding wells from all 6 experimental replicates were plotted (FIGS. 13A-13D, FIGS. 14A-14C).

Statistical analysis of repeatability: To determine the minimum number of channels and repeats required to reach an acceptable confidence level and variation for a substance's effect, the study explored 95% confidence intervals and coefficient of variation (CV) of various parameters as they were randomly sampled for different combinations of repeats. Specifically, the study took random samples of 1 well out of the 6 technical replicates from each of k out of n=6 experiments where k represents 2 to 6 experimental replicates). The study further randomly selected between 4 and 40 individual worms from each well to determine the effect of different sample sizes for individual worms.

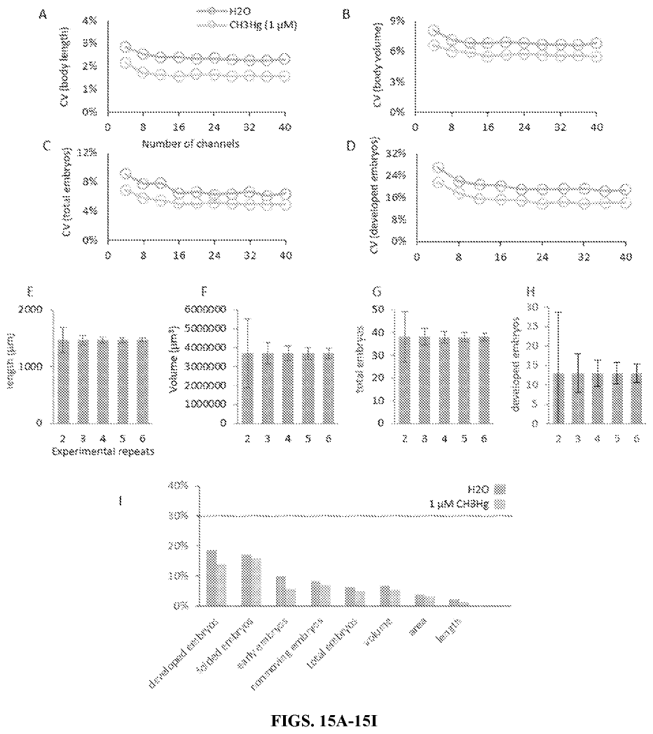

Coefficient of variation decreased with channel number, reaching a plateau before 40 animals (~24 channels) (FIGS. 15A-15D). 95% CI for the mean observed values for each phenotype increased with experimental replicate number. For 2 repeats, the 95% CI around the mean for total embryos was 38.4±18.3 embryos, but reduced to 38.4±5.9 embryos with 3 repeats. Further increases in experiment number reduced the 95% CI further (FIGS. 15E-15H). The study calculated the average CV values for all phenotypes and grouped phenotypes using samples of 3 experimental replicates and 40 channels. All quantitative scores were below the 30% threshold considered to be the maximum acceptable CV value for a comparable toxicology assay (OECD earthworm acute toxicity and reproduction assay #222) in the 1 μM CH₃Hg toxicated growth condition. CV values were also well below the 30% threshold in the control condition for all phenotypes (FIG. 15I). Overall, these results indicated that 3 experimental repeats with each well trapping a minimum of 24 out of the 40 maximum worms would be likely to provide an acceptable confidence level for predicting the effective dose of a test substance.

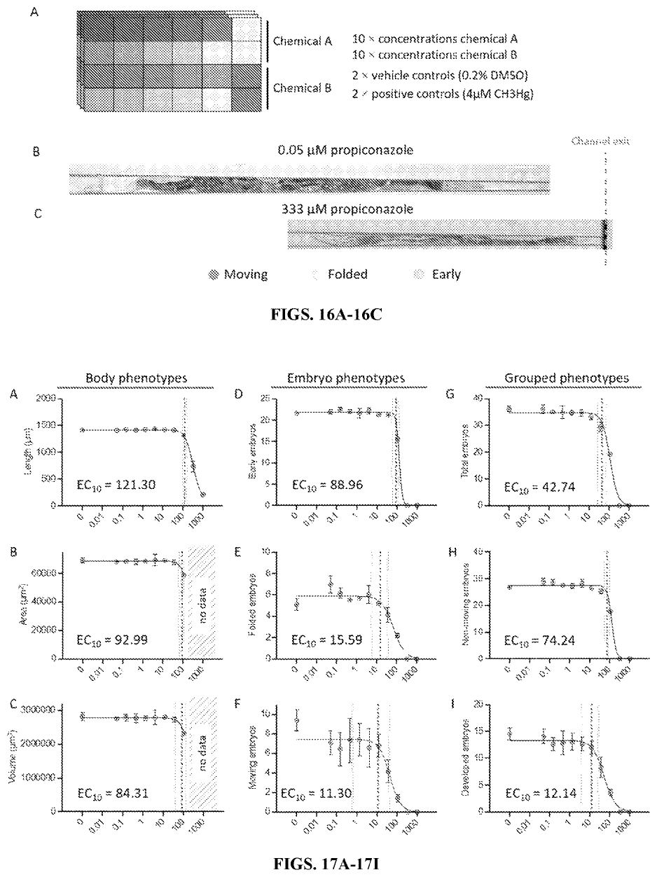

DART case study with an agrichemical in the C. elegans model: Following this demonstration of the assay precision and repeatability, the study performed a dose-response DART study on the fungicide propiconazole (PPZ) [23,24], a chemical of interest to the agrichemical industry and ecotoxicology. The study treated synchronized L1 larvae of each strain following the standard protocol with various doses of the chemical from 0.05-1000 M (in the region of previously published dose-response data) along with 4 μM CH₃Hg as a positive control (FIG. 16A). Adult worms were imaged in the vivoChip-24x after 72 hrs (FIGS. 16B-16C).

The study quantified the individual embryo phenotypes (early, folded, and moving embryos), the grouped phenotypes (non-moving, developed, and total embryos), and body dimensions (length, area, and volume) and calculated the EC₁₀ values and 95% confidence interval bands of each from the fitted Hill function curves (FIGS. 17A-17I). For propiconazole, body length was the least sensitive phenotype, not becoming affected until relatively high concentrations (EC₁₀=117.5 μM, FIG. 17A). At 333 UM or higher, however, the reduced growth rate meant the worms were too small to be immobilized in the microfluidic channels, which were designed for D1 adult animals, so observations of embryo number and length measurements were manually performed using 10× imaging of >10 worms in the well plates. Body volume was a more sensitive readout with an EC₁₀ of 74.9 μM, mostly due to the reduced number of embryos in the body of the worm that was developmentally arrested (FIG. 17C). The effects on embryo number were observed at lower concentrations (EC₁₀=48.8 μM for total embryos). The most sensitive phenotype for propiconazole DART toxicity was the moving embryos, with an EC₁₀ of 10 μM (FIG. 17F). However, a similar EC₁₀=10.9 μM was seen for the developed (moving+folded) embryos with an even tighter confidence interval band (FIG. 17I). These results demonstrate the increased sensitivity of a multiparametric assay that can quantify multiple phenotypes and detect the lowest effective dose for DART.

### DISCUSSION

DART toxicity studies are required for hazard assessment of thousands of new and existing substances, but the high costs and long study durations combined with a global push to end use of animals in routine testing are a major bottleneck in this process. Here, this study demonstrates an imaging-based DART assay using C. elegans which is much cheaper and faster and avoids the use of animals subject to welfare regulation. C. elegans is a complete organism with reproductive features comparable to humans and has been shown to be equally predictive as rodents in some toxicology spaces which gives this assay high potential to be predictive of human DART toxicity.

While C. elegans has been used as a model organism in DART before, previous assays were either low throughput, or used a few gross phenotypes such as brood size or body length. This study sought to develop methods that could detect subtle sublethal DART phenotypes to increase assay sensitivity to detect the effects of substances at low levels while achieving higher throughputs. The study found that high resolution in-utero embryo imaging allowed classification of the F₁ embryos into different developmental stages, the numbers of which were sensitive to toxicants. In utero imaging also removes any confounding effects of chemicals studied on the egg-laying neurons which may affect brood size measurement, as all the embryos studied are still within the germline. Embryo phenotypes, especially the numbers of moving and developed embryos, were more sensitive to the chemicals tested than classic toxicity endpoints such as body length, and so are likely to be more predictive of the lowest levels of toxicants that can cause adverse outcomes.

The study also measured body dimensions (length and volume) as they have been shown to be highly useful and simple to measure developmental endpoints. Current automated methods of body measurement in C. elegans include the COPAS Biosorter and plate readers with image analysis software. However, the COPAS is limited to finding worm length, and while plate readers can measure area using 2D-imaging, they are often performed at low resolutions, limiting accuracy and sensitivity. While methods of measuring C. elegans body volume exist, they are limited to labor intensive manual measurement or complex microfluidic measurements of single worms [26,27]. Since immobilization within the vivoChip microchannels provides high-resolution images of straightened worm bodies contained within channels of known height, an ML-assisted image segmentation could be implemented to find body area and volume of individual worms with high precision, in addition to length. The study found body volume to be more sensitive than length in the study of agrichemicals, and also correlated with total embryo number, indicating this phenotype could be a useful endpoint in developmental toxicity which can be fully automated.

Non-moving (early plus folded embryos) have a lower variation than moving embryos. The high variation of moving embryos could be because of the oscillatory nature of the moving embryo count as they are laid in groups every 1-2 hrs and may be immobilized and imaged before or after an egg laying event. However, if the rate of oocyte production and development remains steady, worms may be more likely to lay eggs if the mechanical pressure in the germline is increased by larger numbers of developing, folded embryos and so the loss of moving eggs as they are laid will be balanced by the increased folded population, leading to lower variation seen in the combined populations.

Intralaboratory (and interlaboratory) repeatability of assays is a vital part of any toxicology assay [28,29]. Towards this end, the study developed a highly robust set of standard operating procedures (SOPs), covering every aspect of the assay from general worm storage and husbandry, through the preparation of batches of toxicants and synchronized L1 larvae, to the collection and analysis of data.

This assay has high statistical power to detect phenotypic changes caused by toxicity. Based on the average observed SD between experiments, the study found a >80% power to identify a 15% change in embryo number using 3 experimental replicates. The scalability of the assay means that this could be increased even further with a larger number of repeats. In addition, the study found that increasing the number of worms used for analysis past 24 did not decrease the CV, supporting the decision to use a 40-channel device and allowing some margin for lower filling efficiencies. The study found a low degree of variance between technical replicates (wells containing worms from the same synchronized batch and the same chemical stock) with an average inter well average CV of 5.83% for total embryo number and 5.67% for body volume. Moving embryo variability was higher, both between individual animals and between well averages, but still acceptable at 26.91%. Because biological variability is higher than technical variability, it is generally considered better to use more biological replicates [30]. The study tested the effect of using different numbers of experimental replicates subsampled from the 6-experiment/6-well dataset and found that for a set of 3 experiments, CV for the embryo phenotypes in control populations was 6.4-18.8% and 2.3-6.9% for body dimensions.

To demonstrate the utility of the assay, the study explored the fungicide and known toxicant propiconazole. The study found that embryonic and developmental phenotypes were affected in a dose dependent manner. Embryonic phenotypes were the most sensitive, especially the moving and developed phenotypes that look at the numbers of late-stage pre-hatching embryos. For propiconazole, the study observed lower EC₁₀ values for moving and developed embryos than all other phenotypes. Both phenotypes gave similar EC₁₀ values, however the variation and confidence interval bands were slightly better for the developed phenotype (moving and folded grouped together). Developmental phenotypes were not as sensitive to the chemical tested, but the body volume did show a lower EC₁₀ value than body length, indicating the volume parameter may be a better indicator of DART toxicity. Better separation between control and treated populations for the volume parameter versus length were also seen in the CH₃Hg repeatability tests.

Other alternative model organisms have been used to study DART effects of propiconazole. Fathead minnow fecundity and Daphnia embryonic development were both affected from 0.5 mg/L (1.46 μM) [31,32]. While the endpoints quantified in those studies are different, the values are close to the lowest EC₁₀ value of 10 μM.

Increasing the numbers of experimental repeats and the dose range of chemicals tested may help improve the predictivity of the assay. In addition, the use of multiple genetically diverse strains may also be of value in detecting toxicity at lower limits. While a single common laboratory strain of model organism may have genetic blindspots reducing sensitivity to certain toxicants, the use of diverse wild-type strains may increase the range of detection, and these resources are widely available for C. elegans [35].

The DART approach developed has the potential to be both cheaper and faster than traditional vertebrate assays. The costs of C. elegans culture and maintenance are vastly lower compared to rodent colonies or fish aquaria. Culture and treatment times are in the range of days due to the short life cycle of worms, and chip loading and imaging times are <1 hour. Analysis can also be performed rapidly. The ML inference pipeline used for body dimension analysis can analyze 1 chip (~1,000 animals) in <30 minutes using a single desktop PC. Comparable timespans could be expected for automated embryo detection using similar ML-based models. The study also showed that the results obtained are highly reproducible over time even when conducted by multiple scientists within a lab over months. Case studies of two agricultural chemicals showed effects on multiple endpoints within the assay, with a range of different effective concentrations for different endpoints with narrow confidence intervals. This range allowed prediction of a minimum effective concentration at which any DART effect was observed for each chemical, highlighting the advantage of a multi-parametric assay.

### Example 2: Automated Analysis of C. elegans Images

Microfluidics technology significantly enhances high-throughput assays in Caenorhabditis elegans research, facilitating the precise control and analysis of immobilized specimens to improve both efficiency and scalability of chemical testing. This breakthrough is particularly valuable in efficacy and toxicity screenings, enabling the in-depth investigation of varied phenotypic responses in C. elegans when exposed to chemicals during early discovery phases. C. elegans models can provide several phenotypes relevant to toxicity studies. This example focused on the developmental and reproductive toxicity (DART)-related endpoints. Conventionally, these phenotypes are scored manually, encountering user bias and being time-consuming, expensive, and low throughputs. Advanced data analysis techniques are clearly necessary to effectively analyze and interpret the complex phenotypes identified in C. elegans for DART assessment. A study was conducted which introduces an automated segmentation framework for C. elegans that reaches human-level accuracy for the task of worm body segments. The study also introduced a framework implemented for organ-level studies, such as embryos inside the C. elegans uterus, to identify adverse health effects associated with the reproductive organ. These approaches facilitate the exploration of phenotypic variations under diverse chemical exposures, showcasing the framework's utility in handling complex data sets and enhancing assay scalability and precision.

High-resolution imaging of C. elegans models: C. elegans strain (N2) is cultured on standard solid medium at 20° C. to obtain sufficient number of gravid worms. Many embryos are collected from the gravid adults by sodium hypochlorite treatment and allowed to develop into synchronized larvae 1 (L1) stage worms overnight. L1s are placed in a 24-well plate with HB101 food in S media. The L1 larvae are treated with known developmental and reproductive toxicants plus untreated controls and incubated at 20° C. for 72 hrs till the day 1 (D1) adult stage.

All 24 populations of D1 worms in M9 buffer were loaded into a 24-well microfluidic device (vivoChip-24x, vivoVerse). Each well contains 40 parallel, gently tapering 3 mm long microfluidic trapping channels, for a total of 960 channels in vivoChip-24x. Once all channels fill up with worms, immobilized inside the narrowing tapered channels, a constant fluid pressure through a gasket system holds them still for performing blur-free imaging. Automated high-resolution imaging is then performed on all 960 channels to collect time-lapse, z-stack images within 30 minutes using a customized automated microscope and controlled using an in-house image-acquisition software (vivoImager). Each well is imaged at 10×, 0.4 NA using 5×8-channel fields of view (FOVs) per well, with 10×6-micron spaced z slices centered around the best focal plane of that cross marker and 5×1-second spaced stacks per FOV to construct a 4D timelapse hyperstack for each FOV. Images are automatically uploaded to a local server for processing and analysis.

For image processing, each channel is cropped into a separate hyperstack. For each cropped specimen, the height of the image is fixed to 5056, and the width is padded to a final width of 384. The central focal plane is found by taking the Laplace of slices from the hyperstack. After identifying the central plane, N focal planes from each side of this central slice are collected and stacked to form the inflated tensor e.g., (2N+1)×H×W. In practice, the study found 3 total z-slices optimal for the model.

Analysis of C. elegans body using 2.5D U-Net model: The model is defined by 2.5D U-Net with an attention mechanism at the bottleneck for the classification and semantic segmentation of C. elegans organs (FIGS. 18A-18C). The proposed architecture includes the following sub-networks: a fully convolutional encoder, a bottleneck layer including a small vision transformer (ViT), and a fully convolutional decoder. This network produces two outputs: a voxel-wise segmentation over C classes produced by the decoder and an image-wise classification over M classes produced at the bottleneck layer. The 2.5D U-Net is similar in design to what Torbunov et. al. [DOI 10.48550/arXiv.2203.02557] proposed for the problem of 2D image generation.

Among all the parameters, 80% are contained within the 2.5D convolutional encoder-decoder that follows the standard U-Net architecture [https://doi.org/10.48550/arXiv.1505.04597]. Unlike a 3D CNN that would convolve volumetric features, the dimension associated with the z-plane is stacked over the feature dimension in a manner similar to how spectral information is treated. The general processing of this architecture is a repetitive layer-wise operation that remains identical between the encoder and decoder. It is a 2D residual convolutional layer and follows the general structure proposed by He et. al. in their work on the ResNet architecture [https://doi.org/10.48550/arXiv.1512.03385]. The final network layer includes linear projections and the softmax function to produce a soft segmentation over C voxel classes.

The remainder of the network is contained within the small ViT introduced to enable efficient, long-range communication between embedded voxels. The output of the ViT is routed to two separate sub-networks: the previously described convolutional decoder as well as a classification subnetwork. The classifier involves a pooled attention mechanism introduced by Lee et. al. with a single seed vector followed by a series of linear layers to produce a vector with M elements such that it can be used for image-wise classification task [https://doi.org/10.48550/arXiv.1810.00825].

The predicted 2D segmentation was used to calculate multiple endpoints, including the length, area, and volume of the C. elegans. Using the calculated 2D segmentation, the longest-spanning tree of the skeleton is calculated to find the length. Using the known chip geometry and the C. elegans position within the device, extrude the 2D mask to a 3D volume. The study found this model to provide an average Dice score of 0.98, a perfect classification accuracy, and an absolute error of less than 1% regarding C. elegans length/volume ratios (TABLE 1). The study compared this model to a standard 2D U-Net, where the ViT is stripped out and only a single z-plane at the best focal plane is used. This approach demonstrates less bias in its length and volume measurements, as indicated by a mean approaching one, and presents a more compact distribution with lower variance.

TABLE 1
Results from the 2.5D U-Net model compared with the standard U-Net model.

| | | | | Classification |
|---|---|---|---|---|
| | Dice Score | Length Ratio | Volume Ratio | Accuracy |
| 2.5D U-Net w/Att | 0.977 ± 0.014 | 0.992 ± 0.018 | 1.001 ± 0.036 | 1.000 ± 0.000 |
| Standard 2D U-Net | 0.958 ± 0.021 | 0.976 ± 0.034 | 1.022 ± 0.056 | 0.981 ± 0.017 |

Analysis of C. elegans in-utero embryos: The study developed an embryo model defined by 2.5D U-Net for the classification and instance segmentation of C. elegans embryos. The proposed architecture includes the following sub-networks: a fully convolutional encoder, a convolutional bottleneck layer, and a fully convolutional decoder. At the decoder, the following outputs are produced: a soft segmentation that denotes the centers of detected embryos, a series of estimated bounding boxes to denote the regions of interest, a score that estimates the likelihood that an embryo lies within the region of interest, and a mask if an embryo is scored to be present within the specified region of interest. The scores produced by the model identify the probability of an object and assigns the relevant class to non-background objects. This is achieved by applying the softmax function to a vector with C+1 elements.

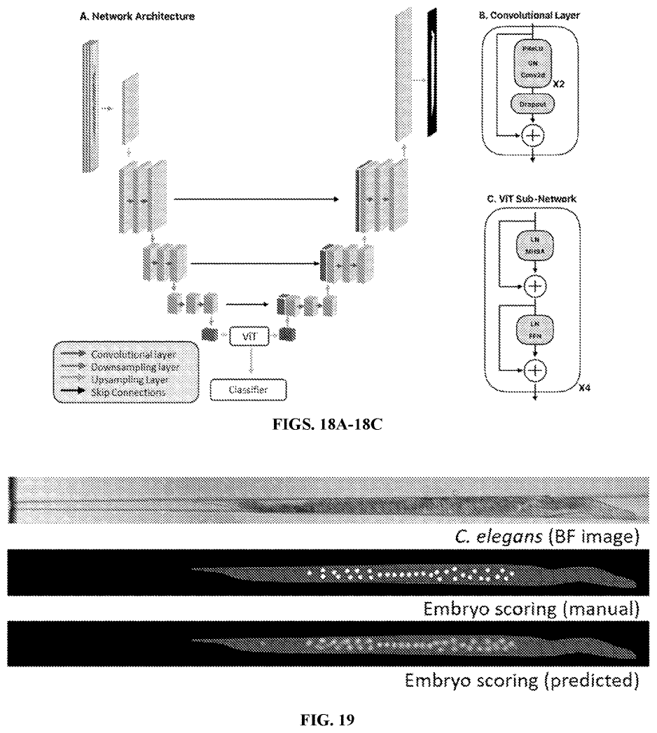

In full, this model represents a convolutional, two-stage object detector. Unlike a 3D CNN that would convolve volumetric features, the dimension associated with the z-plane is stacked over the feature dimension. This is similar in design to how hyper-spectral data is often treated. The general processing of this architecture is a repetitive layer-wise operation that remains identical between the encoder and decoder. It is a 2D residual convolutional layer and follows the general structure proposed by He et. al. in their work on the ResNet architecture. Following the decoder, multiple specialist subnetworks are introduced that produce all the components of a 2-stage model (center estimates, bounding boxes, scores, and intra-bounding box segmentations). These specialist subnetworks are kept identical to one another in design. They all include a multi-layer perceptron to align the decoder features to the task at hand and a series of linear layer to regress this vector onto the target. Calculated endpoints include the total number of embryos (FIG. 19) and embryo classes. Total embryos are determined by counting instances of bounding boxes classified as containing an embryo where the classes are directly provided by the model.

To elucidate different mechanisms of action, we can use transgenic strains to visualize the chromatin organization and defects in the embryonic development of C. elegans using vivoChip. For example, high-resolution images of the entire 3-dimensional (3D) germline in C. elegans can be visualized using two-color fluorescence reporters (FIGS. 20A-20E). The images can allow us to analyze the defects in the chromatin organization and identify dead and viable embryo populations using the time-lapse imaging of immobilized animals.

### Example 3: Analysis of C. elegans and Internal Organs Using Different Strains and Fluorescent Dyes

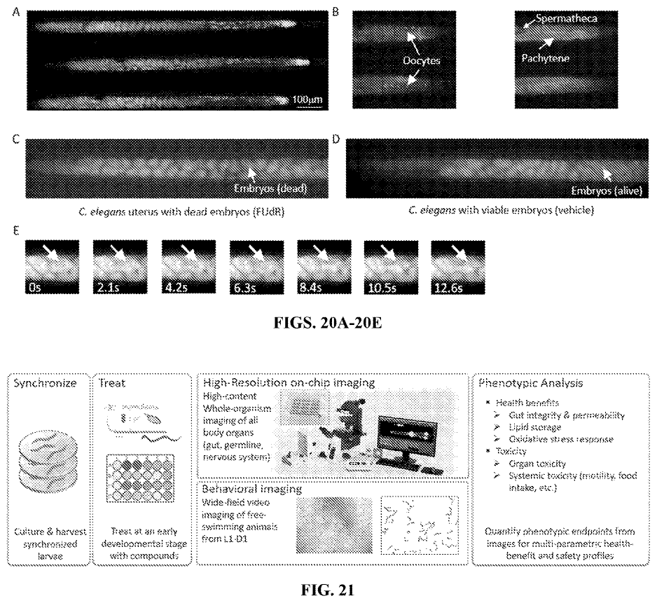

A study was conducted to analyze the body and internal organs of C. elegans using high-resolution imaging techniques, bright field images, fluorescent dyes, or fluorescent reporters. FIG. 21 shows an overview of the study.

Liquid culture of C. elegans: Worms are synchronized by bleaching a population of gravid C. elegans using the hypochlorite solution. The embryos are then incubated in a glass tube and rotated for 24 hours at 20° C. About 80 larval 1 (L1) worms are plated into a standard 24-well plastic plate with 500 μl S media and HB101 bacteria. The worms are exposed to chemicals or ingredients during the growth in the liquid media for 3 days in a 20° C. incubator.

Imaging of C. elegans cultures: C. elegans populations are grown in liquid media using standard 24-well plates. The culture wells are imaged at different time points during their development. For example, this report presents the bright-field images of the 24-well plates with ~80 worms per well and fed with HB101 E. coli bacteria. The worms in individual wells are grown with different chemicals and at a specific dose, including positive and negative controls. The well plates with worms and bacteria are captured using 2×, 0.08 NA objective (FIGS. 22A-22B). The images are used to quantify bacteria density, worm size, and worm movement to analyze parameters such as food intake (or feeding rate), worm development, and motility.

Imaging of C. elegans using vivoChip: The C. elegans are fed with bacteria and treated with test chemicals for 72 hours in a 20° C. incubator. At 72 hours, when the control populations are in the adult stage of day 1 (D1), all 24 populations are transferred to a vivoChip-24x device using a multi-channel pipette. The worms are immobilized inside the microfluidic channels under each well for high-resolution imaging (FIG. 22C). The vivoChip technology allows capture of bright field or fluorescence images from the worms immobilized inside the microfluidic channels.

Image analysis of the C. elegans body and fluorescence signal: The images from the vivoChip platform were cropped into individual channels. The study determined the best focal plane for each worm image (bright-field images) from the stack of images. Individual channel image was analyzed using machine-learning algorithms (developed by vivoVerse) to predict the mask for the worm body inside the individual microfluidic channel. Since several organs in the worm body have a 3D architecture, the study created a maximum-intensity projection of multiple z-slice fluorescence images. Using the maximum projection image, the study then calculated the integrated fluorescence signal within the predicted body mask. The study also estimated the background signal from the pixels outside the body mask and within two successive channel regions. The background signal was used to estimate the corrected total C. elegans fluorescence (CTCF)=integrated density−(area of selected C. elegans body×fluorescence of background readings)

Nile red (NR) staining to study body lipids: To stain lipid molecules in the C. elegans body, 30 ng/ml and 50 ng/mL were added to the liquid culture at the L1 stage (a total of 72-hour incubation) and L4 stage animals (a total of 24-hour incubation) and maintained the plate at 20° C. A well with no dye was used as the control population. At the end of 72 hours, the worm populations were loaded into the vivoChip and imaged using a 10×, 0.4 NA objective. Bright-field and fluorescence (red filter set) were acquired to capture worm development and the stored lipids in the C. elegans populations.

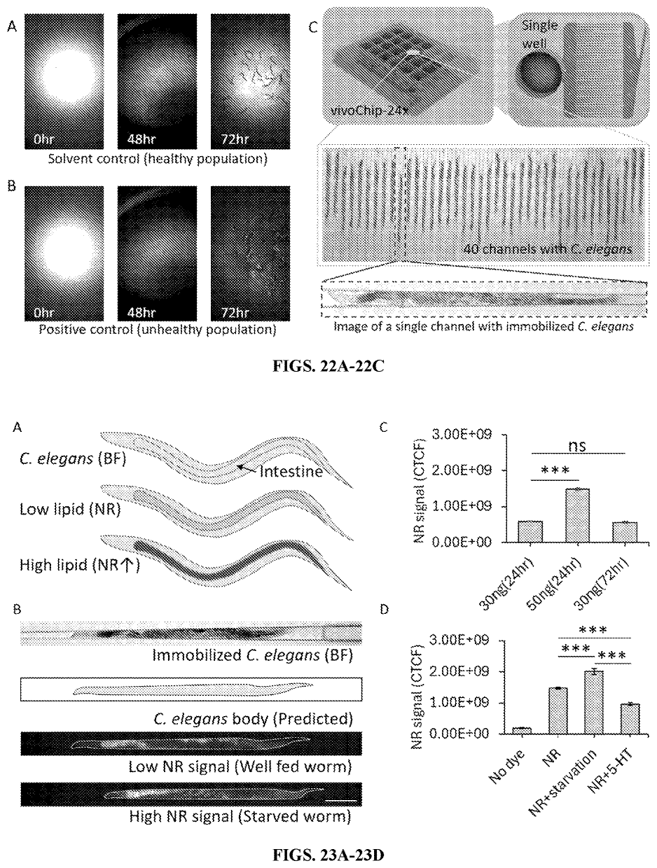

Analysis of NR signal: The individual channels were analyzed to identify the NR signal, mainly within the worm intestinal muscle (FIG. 23A). Since the intestine has a 3D architecture in the worm body, the study created a maximum intensity projection of all 10 z-slice, red-fluorescence images. The study then calculated the integrated red fluorescence signal within the ML-predicted body mask using the maximum projection image (FIG. 23B).

The NR signal from the C. elegans population treated with 50 ng/ml for 24 hours was 2.5× higher than the 30 ng/mL (p-value <0.001, FIG. 23C). The staining with 30 ng/mL for a longer duration (72 hours) did not show a statistically significant increase in the integrated intensity. To test the efficacy of the assay, the study induced starvation by removing the worms from the food at 68 hours. The worms were filtered using a 40-micron mesh filter and replated in a fresh well with no food in S media with NR dye. The worms were then imaged at 72 hours to identify the NR signal. The starved worms showed a significant increase in the NR signal (2.02×109±0.09×109), which is significantly different (p-value <0.001) from the well-fed worm population (FIG. 23D). This data agrees with previously published data. As shown by previous studies, serotonin (5-HT) also affects lipid metabolism. The worms were co-cultured with 5-HT for the entire 72 hours and found that they had a significantly lower amount of lipids in their body, visualized using NR staining.

Induction of oxidative stress and analysis of gst-4::GFP reporter: Like other model systems, C. elegans encounters exogenous and endogenous stressors, including oxidative stress, causing physiological changes in the C. elegans. The C. elegans genome has conserved transcription factors such as DAF-16/FOXO, SKN-1/Nrf2, and detoxification genes (gst-4 and sod-3) and their associated roles in response to oxidative stress responses. The study treated a transgenic strain expressing GFP when exposed to oxidative stress to quantify the stress level in individual worms using high-resolution imaging to quantify sub-lethal changes in the stress levels.

The study used the CL2166 strain (obtained from CGC) that expressed a GPF signal when oxidative stress was induced using hydrogen peroxide (H₂O₂) in culture media to study oxidative stress. 5.66 μl of 3% H₂O₂ was added to the 500 μl S media culture with worms at 68 hours to induce oxidative stress. The worms were incubated for another 4 hours inside a 20° C. incubator before being imaged for high-resolution imaging and analysis.

The CL2166 worms had a baseline GFP intensity in the absence of any treatment. The GFP intensity increased by ~60% upon H₂O₂ exposure (FIG. 24A, FIG. 24C). The BF worms showed no changes, while the GFP signal in the H₂O₂-treated population was higher (FIG. 24B, FIG. 24D).

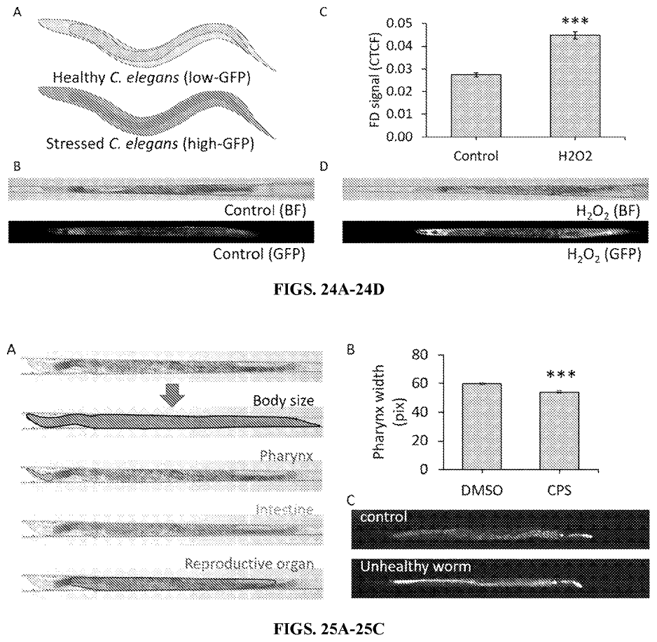

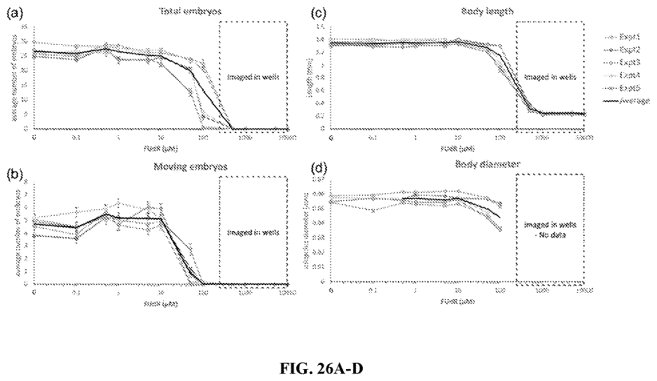

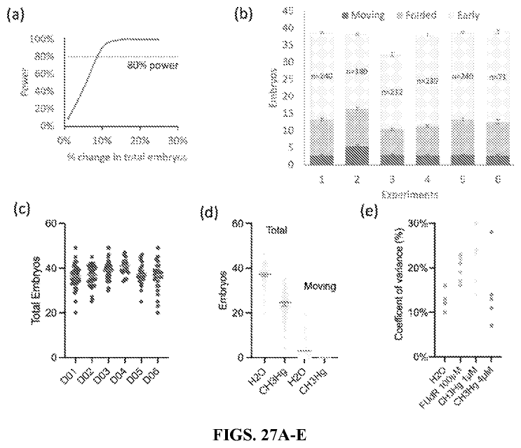

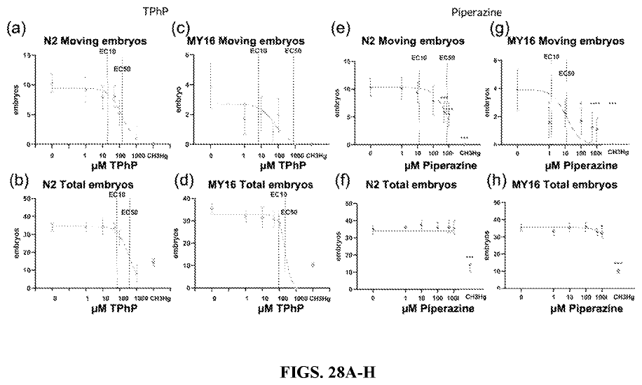

Identifying internal organs and organ structures of C. elegans using bright field and fluorescence images: Overall morphology of changes in different tissues within a C. elegans body. Due to the dynamic nature of the tissue architectures, it is expected to change over the animal's age. This approach was used to track tissue architecture during early adult stage C. elegans quantitatively (FIG. 25A). As an example, the study analyzed the pharynx within the C. elegans immobilized inside the vivoChip. The pharynx is involved in the pumping action, which is controlled by the pharyngeal motor neurons and the release of acetylcholine. This well-studied organ has been used to screen several therapeutic drugs and to characterize the amount of food intake readouts. The pharyngeal architecture also changes when the animals are exposed to toxic chemicals.

C. elegans pharynxes present in the best focal plane from individual channels were manually segmented. The images were used to train a network to identify the pharynx automatically. The total number of pixels capturing individual pharynx was used to determine the worm's health. The total pixels were reduced in animals treated with chlorpyrifos (CPS) compared to the vehicle control (DMSO) as shown in FIG. 25B. Once the C. elegans body is detected, specific organs can be characterized using the fluorescence reporter (transgenic strains) or fluorescent dyes (feeding or soaking with dye). For example, C. elegans were fed with FITC-dextran overnight to visualize the green signal using fluorescence imaging of device-immobilized animals. The high-resolution images allow quantification of the dye inside the intestinal lumen. Combining the fluorescence-based analysis with organ identification, the amount of dye inside the body and its localization can be determined to characterize important health benefit-related parameters such as bioavailability, leaky gut, and localization.

### Example 4: Using C. elegans for Rapid, Cost-Effective In Vivo Toxicology Assessments

The whole-body fluorescence imaging of C. elegans at high resolutions enables sub-cellular phenotyping of the entire nervous system at a single-neuron level. The health of each neuron can be analyzed at sub-lethal doses by scoring multiple axonal/dendritic degeneration phenotypes such as beading, breaks, deformation, and tip loss. Specifically, we used a dopaminergic neuronal reporter strain of C. elegans (DAT-1::GFP) to quantify the developmental neurotoxicity (DNT) of chemicals. Among eight dopaminergic neurons, the four cephalic sensilla (CEP) and two ADE neurons are especially well-suited for neurotoxicity assessment. Their proximity to the mouth makes them among the first neurons exposed to the toxicants. We, therefore, assessed these neurons using 3D fluorescence images of immobilized C. elegans in our vivoChip after they were exposed to various doses of a well-characterized neurotoxicant and found a dose-dependent increase in multiple phenotypes (FIG. 29).

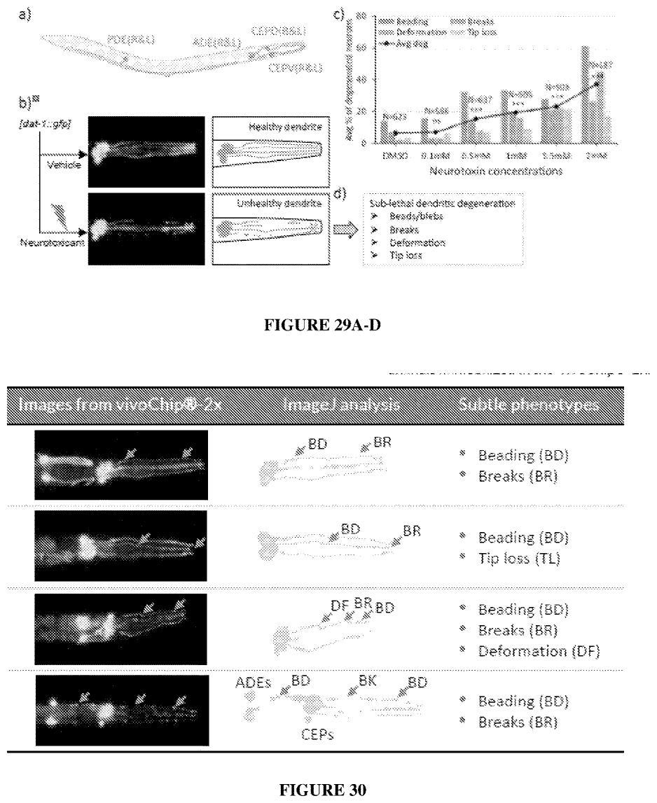

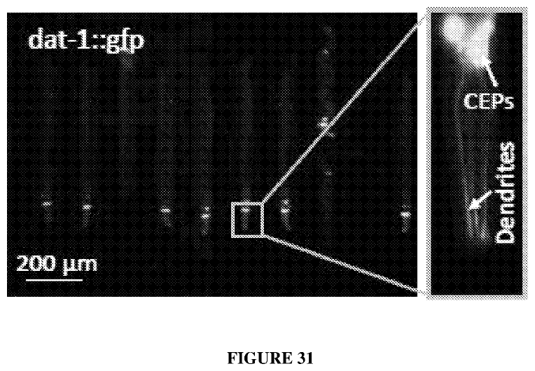

Neuro-degeneration studies in C. elegans require high resolution imaging of a large number of immobilized animals (FIG. 30). The vivoChip® enables detection of sub-lethal, multiparametric, and neuron-specific phenotypic changes in 40 dat-1::gfp labeled animals at once (FIG. 31). Up to 40 adult animals were immobilized in the vivoChip®-2x within 3 min. Dopaminergic neurons labeled with dat-1::gfp are imaged at high magnifications (20×, 0.75NA). Neuronal degenerations are induced in adult animals by treatment with the neurotoxinMPP+iodide. Defined classes of structural aberrations (phenotypes) are evaluated in the immobilized animals.

The following patents, applications and publications as listed below and throughout this document are hereby incorporated by reference in their entirety herein.

### REFERENCE LIST

1. Wang Z, Walker G W, Muir D C G, Nagatani-Yoshida K. Toward a Global Understanding of Chemical Pollution: A First Comprehensive Analysis of National and Regional Chemical Inventories. 2020; doi: 10.1021/acs.est.9b06379
2. Huang C Y, Nicholson M W, Wang J Y, Ting C Y, Tsai M H, Cheng Y C, et al. Population-based high-throughput toxicity screen of human iPSC-derived cardiomyocytes and neurons. Cell Rep. Cell Press; 2022; 39:110643. doi: 10.1016/J.CELREP.2022.110643
3. Brannen K C, Chapin R E, Jacobs A C, Green M L. Alternative Models of Developmental and Reproductive Toxicity in Pharmaceutical Risk Assessment and the 3Rs. ILAR J. ILAR J; 2016; 57:144-156. doi: 10.1093/ILAR/ILW026
4. Chahardehi A M, Arsad H, Lim V. Zebrafish as a Successful Animal Model for Screening Toxicity of Medicinal Plants. Plants. Multidisciplinary Digital Publishing Institute (MDPI); 2020; 9:1-35. doi: 10.3390/PLANTS9101345
5. Gutner-Hoch E, Martins R, Maia F, Oliveira T, Shpigel M, Weis M, et al. Toxicity of engineered micro- and nanomaterials with antifouling properties to the brine shrimp Artemia salina and embryonic stages of the sea urchin Paracentrotus lividus. Environ Pollut. Elsevier; 2019; 251:530-537. doi: 10.1016/J.ENVPOL.2019.05.031
6. Tkaczyk A, Bownik A, Dudka J, Kowal K, Ślaska B. Daphnia magna model in the toxicity assessment of pharmaceuticals: A review. Sci Total Environ. Sci Total Environ; 2021; 763. doi: 10.1016/J.SCITOTENV.2020.143038
7. Lai C-H, Chou C-Y, Ch'ang L-Y, Liu C-S, Lin W-C. Identification of Novel Human Genes Evolutionarily Conserved in Caenorhabditis elegans by Comparative Proteomics. Available: www.genome.org
8. Larigot L, Mansuy D, Borowski I, Coumoul X, Dairou J. Cytochromes P₄₅₀ of Caenorhabditis elegans: Implication in Biological Functions and Metabolism of Xenobiotics. 2022; doi: 10.3390/biom
9. Harlow P H, Perry S J, Stevens A J, Flemming A J. Comparative metabolism of xenobiotic chemicals by cytochrome P450s in the nematode Caenorhabditis elegans. Sci Reports 2018 81. Nature Publishing Group; 2018; 8:1-8. doi: 10.1038/s41598-018-31215-w
10. Ben-Yakar A. High-content and high-throughput in vivo drug screening platforms using microfluidics. Assay Drug Dev Technol. 2019; 17:8-13. doi: 10.1089/adt.2018.908
11. Hunt P R, Camacho J A, Sprando R L. Caenorhabditis elegans for predictive toxicology. Curr Opin Toxicol. Elsevier; 2020; 23-24:23-28. doi: 10.1016/J.COTOX.2020.02.004
12. Boyd W A, Smith M V., Co C A, Pirone J R, Rice J R, Shockley K R, et al. Developmental effects of the ToxCast™ phase I and phase II chemicals in Caenorhabditis elegans and corresponding responses in Zebrafish, Rats, and Rabbits. Environ Health Perspect. Public Health Services, US Dept of Health and Human Services; 2016; 124:586-593. doi: 10.1289/EHP.1409645
13. Li Y, Gao S, Jing H, Qi L, Ning J, Tan Z, et al. Correlation of chemical acute toxicity between the nematode and the rodent The utility of any non-rodent model system for chemical toxicity screening depends on the level of corre. Cite this Toxicol Res. 2013; 2:403. doi: 10.1039/c3tx50039j
14. Brenner S. The Genetics of CAENORHABDITIS ELEGANS. Genetics. Oxford University Press; 1974; 77:71. doi: 10.1093/GENETICS/77.1.71
15. Mondal S, Hegarty E, Martin C, Gökçe SK, Ghorashian N, Ben-Yakar A. Large-scale microfluidics providing high-resolution and high-throughput screening of Caenorhabditis elegans poly-glutamine aggregation model. Nat Commun. 2016; 7. doi: 10.1038/ncomms13023
16. Mondal S, Hegarty E, Sahn J J, Scott L L, Gökçe SK, Martin C, et al. High-Content Microfluidic Screening Platform Used to Identify 62R/Tmem97 Binding Ligands that Reduce Age-Dependent Neurodegeneration in C. elegans SC-APP Model. ACS Chem Neurosci. American Chemical Society; 2018; 9:1014-1026. doi: 10.1021/acschemneuro.7b00428
17. Chisholm A D, Hardin J. Epidermal morphogenesis. [Internet]. WormBook: the online review of C. elegans biology. WormBook; 2005. pp. 1-22. doi: 10.1895/wormbook.1.35.1
18. Ardiel E L, Lauziere A, Xu S, Harvey B J, Christensen R, Nurrish S, et al. Stereotyped behavioral maturation and rhythmic quiescence in C. elegans embryos. Elife. eLife Sciences Publications Ltd; 2022; 11. doi: 10.7554/ELIFE.76836
19. OECD/OCDE 407 OECD GUIDELINES FOR THE TESTING OF CHEMICALS Repeated Dose 28-Day Oral Toxicity Study in Rodents INTRODUCTION.
20. Lionaki E, Tavernarakis N. Assessing aging and senescent decline in Caenorhabditis elegans: cohort survival analysis. Methods Mol Biol. Methods Mol Biol; 2013; 965:473-484. doi: 10.1007/978-1-62703-239-1_31
21. Van Der Voet M, Teunis M, Louter-Van De Haar J, Stigter N, Bhalla D, Rooseboom M, et al. Towards a reporting guideline for developmental and reproductive toxicology testing in C. elegans and other nematodes. doi: 10.1093/toxres/tfab109
22. Boyd W A, Smith M V., Kissling G E, Freedman J H. Medium- and high-throughput screening of neurotoxicants using C. elegans. Neurotoxicol Teratol. 2010; 32:68-73. doi: 10.1016/j.ntt.2008.12.004
23. Orlandini Costa N, Leivas Vicira M, Sgarioni V, Rangel M, Percira F, Montagnini B G, ct al. Evaluation of the reproductive toxicity of fungicide propiconazole in male rats. 2015; doi: 10.1016/j.tox.2015.06.011
24. Svanholm S, Säfholm M, Brande-Lavridsen N, Larsson E, Berg C. Developmental reproductive toxicity and endocrine activity of propiconazole in the Xenopus tropicalis model. Sci Total Environ. Elsevier; 2021; 753:141940. doi: 10.1016/J.SCITOTENV.2020.141940
25. Peer review of the pesticide risk assessment of the active substance mesotrione. EFSA J. Wiley; 2017; 14. doi: 10.2903/J.EFSA.2016.4419
26. Bravo F V, Da Silva J, Chan R B, Di Paolo G, Teixeira-Castro A, Oliveira T G. Phospholipase D functional ablation has a protective effect in an Alzheimer's disease Caenorhabditis elegans model. Sci Reports 2018 81. Nature Publishing Group; 2018; 8:1-12. doi: 10.1038/s41598-018-21918-5
27. Dong X, Song P, Liu X. An Automated Microfluidic System for Morphological Measurement and Size-Based Sorting of C. elegans. IEEE Trans Nanobioscience. 2019; 18:373. doi: 10.1109/TNB.2019.2904009
28. Fischer M, Belanger S E, Berckmans P, Bernhard M J, Bl aha L, Coman Schmid D E, et al. Repeatability and Reproducibility of the RTgill-W1 Cell Line Assay for Predicting Fish Acute Toxicity. Toxicol Sci. 2019; 169:353-364. doi: 10.1093/toxsci/kfz057
29. Hoss S, Ahlf W, Bergtold M, Bluebaum-Gronau E, Brinke M, Donnevert G, et al. Interlaboratory comparison of a standardized toxicity test using the nematode Caenorhabditis elegans (ISO 10872). Environ Toxicol Chem. John Wiley & Sons, Ltd; 2012; 31:1525-1535. doi: 10.1002/ETC.1843
30. Blainey P, Krzywinski M, Altman N. Points of significance: Replication. Nat Methods. Nature Publishing Group; 2014; 11:879-880. doi: 10.1038/NMETH.3091
31. Skolness S Y, Blanksma C A, Cavallin J E, Churchill J J, Durhan E J, Jensen K M, et al. Propiconazole Inhibits Steroidogenesis and Reproduction in the Fathead Minnow (Pimephales promelas). doi: 10.1093/toxsci/kft010
32. Kast-Hutcheson K, Rider C V., LeBlanc G A. The fungicide propiconazole interferes with embryonic development of the crustacean Daphnia magna. Environ Toxicol Chem. John Wiley & Sons, Ltd; 2001; 20:502-509. doi: 10.1002/ETC.5620200308
33. Wang C, Harwood J D, Zhang Q. Oxidative stress and DNA damage in common carp (Cyprinus carpio) exposed to the herbicide mesotrione. Chemosphere. Pergamon; 2018; 193:1080-1086. doi: 10.1016/J.CHEMOSPHERE.2017.11.148
34. Elskus A A, Myers M D. In Cooperation with the Maine Department of Environmental Protection Pilot Study of Sublethal Effects on Fish of Pesticides Currently Used and Proposed for Use on Maine Blueberries Open-File Report 2007-1110. Available: http://www.usgs.gov/pubprod
35. Widmayer S J, Crombie T A, Nyaanga J N, Evans K S, Andersen E C. C. elegans toxicant responses vary among genetically diverse individuals. Toxicology. Elsevier; 2022; 479:153292. doi: 10.1016/J.TOX.2022.153292

What is claimed is:

1. A method of analyzing internal components of an organism, the method comprising a) taking an image of internal components of the organism, while said internal components are contained in vivo, and b) analyzing the internal components.

2. The method of claim 1, wherein said method is used to assess toxicity or efficacy of a substance.

3. The method of claim 2, wherein the substance is applied to the organism before imaging of the organism.

4. The method of claim 3, wherein the substance is applied in the gas phase or in the liquid phase.

5. The method of claim 4, wherein when applied in the gas phase, the substance is applied in the chambers or headspace.

6. The method of claim 4, wherein when applied in the liquid phase, the substance is applied in the culture media or absorbed onto the solid media.

7. The method of any one of claims 1-6, wherein the organism is Caenorhabditis elegans, parasitic nematode, roundworm, C. briggsae, zebrafish, daphnia, planaria, and drosophila.

8. The method of any one of claims 1-7, wherein the internal component is a reproductive/germline component.

9. The method of any one of claims 1-7, wherein the internal component is an organ, such as those from digestive, reproductive, nervous, sensory, muscle, or excretory system, pharynx or intestine.

10. The method of claim 8, wherein the reproductive component is a gamete, embryo, stem cell, or chromosome, for example.

11. The method of claim 10, wherein the gamete is an egg, oocyte, or sperm.

12. The method of claim 8, wherein the reproductive component is a fertilized egg.

13. The method of claim 8, wherein the reproductive component is an embryo.

14. The method of any one of claims 1-13, wherein the organism is a hermaphrodite, female, or a male.

15. The method of any one of claims 1-14, wherein the organism is at least partially immobilized prior to imaging or remains free-moving.

16. The method of any one of claims 1-15, wherein organisms can be in a conventional multi-well plate, agar plate, liquid culture, on solid media such as nematode growth media (NGM), gel, polymer, microfluidic environment or microfluidic device.

17. The method of claim 15 wherein the organism is at least partially immobilized using a physical confinement.

18. The method of claim 17, wherein the physical confinement comprises gel, beads, microfluidic device, or other means for immobilization.

19. The method of claim 16, wherein the microfluidic chip is a multi-well plate.

20. The method of any one of claims 1-19, wherein a microscope or imager is used to take the image.

21. The method of claim 20, wherein the microscope or imager is a wide-field microscope, phase microscope, confocal microscope, fluorescence microscope, non-linear microscope, structured-illumination microscope, polarized microscope, computational microscope, plate imager, microscope with point-spread function engineering, or flow-based imager.

22. The method of claim 13, wherein the embryo is in different stages of development such as an early-stage, folded-stage, or moving stage.

23. The method of claims 1-22, wherein the internal component is measured to determine whether it is alive, dead, and/or functioning and to determine its health state and toxicity state.

24. The method of claim 23, wherein functionality of internal components are determined by various parameters and scoring different phenotypes and their classifications.

25. The method of claim 24, wherein volume, size, and/or relative positions of whole body or individual organs, organ contents, and/or their texture are evaluated.

26. The method of claim 25, wherein automated image analysis, machine vision, and/or machine-learning algorithms is used to characterize and classify organs.

27. The method of any one of claims 1-26, wherein dynamical events, such as movement, of the internal component is measured.

28. The method of claim 8, wherein growth/progression of the reproductive/germline component is measured as a function of time.

29. The method of claim 8, wherein malformation or slowed progress in formation of the reproductive/germline component is measured.

30. The method of any one of claims 1-29, wherein more than one organism is used in the same assay.

31. The method of claim 30, wherein the organisms are the same type of organisms or different organisms.

32. The method of claim 30 or 31, wherein the organisms are age synchronized.

33. The method of any one of claims 1-32, wherein time-lapse images are acquired.

34. The method of any one of claims 1-33, wherein the images are analyzed to determine phenotypes relevant to developmental and reproductive toxicity.

35. The method of any one of claims 1-33, wherein the images are analyzed to determine phenotypes relevant to neurotoxicity.

36. The method of claim 35, wherein neurons are analyzed.

37. The method of claim 36, wherein the neuron belongs to a specific neurotransmitter, wherein said neurotransmitter comprises GABAergic, Dopaminergic, serotonergic, or Cholinergic neurons.

38. The method of any one of claims 1-37, wherein the organism has been genetically engineered.

39. The method of any of claims 1-38, wherein the internal component is the intestine.

40. The method of claim 39, wherein the intestinal permeability of the organism is assessed.

41. The method of claim 40, wherein the method of assessing intestinal permeability is measuring the uptake of a colored or fluorescent dye from the intestinal lumen.

42. The method of any of claims 1-38, wherein the internal component is muscle.

43. The method of claim 42, wherein the muscle expresses a fluorescent reporter gene.

44. The method of claim 42, wherein the fluorescent reporter gene marks protein aggregation.

45. The method of claim 42, wherein the fluorescent reporter gene marks oxidative stress.

46. The method of any of claims 1-38, wherein the internal components are lipid storage structures.

47. The method of claim 46, wherein the lipid storage structures are stained with a dye marking specific lipid subtypes.

48. A method of determining toxicity or efficacy of a substance, the method comprising:
a. exposing an organism to one or more substances of interest;
b. taking one or more images of one or more internal components of the organism,
c. analyzing internal components of the organism from the images to determine the effects of the substance on the organism, and;
d. using the results of the analysis to determine the toxicity or efficacy of the one or more substances.

49. The method of claim 48, wherein the substance is applied to the organism in liquid media.

50. The method of claim 48, wherein the substance is applied to the organism absorbed into solid media.

51. The method of claim 48, wherein the substance is applied to the organism in the gas phase or headspace of liquid media.

52. The method of claim 48, wherein the organism is partially or fully immobilized prior to imaging.

53. The method of claim 52, wherein the partial or full immobilization is performed in a microfluidic device.

54. The method of claim 53, wherein the microfluidic device comprises a plurality of features for the purposes of immobilizing multiple organisms simultaneously.

55. The method of either of claim 53, wherein the microfluidic device comprises a plurality of individual isolated compartments to contain multiple separate populations of organisms.

56. The method of claim 48, wherein the imaging step is performed using a microscope.

57. The method of claim 56, wherein the microscope is a wide-field microscope, phase microscope, confocal microscope, fluorescence microscope, non-linear microscope, structured-illumination microscope, polarized microscope, computational microscope, plate imager, microscope with point-spread function engineering, or flow-based imager.

58. The method of claim 48, wherein the imaging step includes taking multiple images of the same organism over time, in order to analyze a dynamic system over time.

59. The method of any of claims 48-58, wherein the internal component is an organ.

60. The method of claim 59, wherein the organ is selected from the group comprising intestine, muscle, germline, uterus, pharynx, or nervous system.

61. The method of any of claims 48-58, wherein the internal component is an organ structure

62. The method of claim 61, wherein the organ structure is selected from the group including intestinal lumen, muscle fiber, lipid granules, or neurons.

63. The method of any of claims 48-58, wherein the internal component is a reproductive component.

64. The method of claim 63, wherein the reproductive component is an egg, sperm, zygote, gamete, germ cell, or embryo.

65. The method of claim 64, wherein the embryo is developed or partially developed.

66. The method of claim 65, wherein the developmental stage of the embryo is assessed.

67. The method of any of claims 48-58, wherein the internal component is the intestine.

68. The method of claim 67, wherein the intestinal permeability of the organism is assessed.

69. The method of claim 67, wherein the method of assessing intestinal permeability is measuring the uptake of a colored or fluorescent dye from the intestinal lumen.

70. The method of any of claims 48-58, wherein the internal component is muscle.

71. The method of claim 70, wherein the muscle expresses a fluorescent reporter gene.

72. The method of claim 71, wherein the fluorescent reporter gene marks protein aggregation.

73. The method of claim 71, wherein the fluorescent reporter gene marks oxidative stress.

74. The method of any of claims 48-58, wherein the internal components are lipid storage structures.

75. The method of claim 64, wherein the lipid storage structures are stained with a dye marking specific lipid subtypes.

76. The method of any one of claims 48-75, wherein automated image analysis, machine vision, and/or machine-learning algorithms is used to characterize and classify organs.

77. The method of any one of claims 48-75, wherein manual analysis is used to classify organs.
